SEQUENCE LISTING (88 sequences)

SEQ ID NO: 1 - protein, 239 aa, Homo sapiens
MRLPAQLLGLLMLWVPGSSGDIVMTQTPLSSPVTLGQPASISCRSSQSLVHSDGNTYLNWLQQRPGQPPRLLIYKISNRFSGVPDRFTGSGAGTDFTLKISRVEAEDVGVYTCMQVTQFPLTFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 2 - protein, 470 aa, Homo sapiens [sequence omitted]

SEQ ID NO: 3 - protein, 235 aa, Homo sapiens
METPAQLLFLLLLWLPDTTGEIVLTQSPGTLSLSPGDRATLSCRASQSVSSSYFAWYQQKPGQAPRLLIYGASSRATGIPDRFSGSGSGTDFTLTISRLEPEDFAVYYCQQYDRSPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 4 - protein, 463 aa, Homo sapiens
MDWTWRILFLVAAATGAHSQVQLVQSGAEVKKPGASVKVSCKSSGYTFTGYYMHWVRQAPGQGLEWMGWINPNSGGTNYAQKFKGRVTMTRDTSISTAYMELSRLRSDDTAVYYCARDKWLDGFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 5 - protein, 235 aa, Homo sapiens
MAWALLLLTLLTQDTGSWAQSVLTQSPSASGTPGQRVTISCSGSSSNIGSNYVYWYQQLPGAAPKLLILRNNQRPSGVPDRFSGSKSGTSASLTISGLRSEDEADYYCAAWDDSLSGWVFGGGTKLTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 6 - protein, 479 aa, Homo sapiens
MDMRVPAQLLGLLLLWLRGARCEVQLVESGGGLVKPGGSLRLSCAASGFTFSNAWMSWVRQAPGKGLEWVGRIKSKTDGGTTDYTAPVKGRFTISRDDSKNTLYLQMNSLKAEDTAVYYCTTDRTGYSISWSSYYYYYGMDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 7 - protein, 235 aa, Homo sapiens
METPAQLLFLLLLWLPDTTGEIVLTQSPGTLSLSPGERATLSCRASQSVRGRYLAWYQQKPGQAPRLLIYGASSRATGIPDRFSGSGSGTDFTLTISRLEPEDFAVFYCQQYGSSPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 8 - protein, 467 aa, Homo sapiens
MEFGLSWLFLVAILKGVQCEVQLLESGGGLVQPGGSLRLSCAASGFTFSSYAMSWVRQAPGKGLEWVSGITGSGGSTYYADSVKGRFTISRDNSKNTLYLQMNSLRAEDTAVYYCAKDPGTTVIMSWFDPWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 9 - protein, 242 aa, Homo sapiens
MDMRVPAQLLGLLLLWLRGARCDIVMTQTPLSLPVTPGEPASISCRSSQSLLNSVDGSTNLDWYLQKPGQSPQLLIYTLSYRASGVPDRFSGSGSGTDFTLKISRVEAEDVGVYYCMQRIEFPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 10 - protein, 465 aa, Homo sapiens
MELGLCWVFLVAILEGVQCEVQLVESGGGLVQPGGSLRLSCAASGFTFSSYSMNWVRQAPGKGLEWVSYISSSGSSIYYADSVKGRFTISRDNAKNSLYLQMNSLRDEDTAVYYCARERYYGDTPFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 11 - protein, 233 aa, Homo sapiens
MSPSQLIGFLLLWVPASRGEIVLTQSPDFQSVTPKEKVTITCRASQSIGSSLHWYQQKPDQSPKLLIKYASQSFSGVPSRFSGSGSGTDFTLTINSLEAEDAAAYYCHQSSSLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 12 - protein, 463 aa, Homo sapiens
MGSTAILALLLAVLQGVCAEVQLMQSGAEVKKPGESLKISCKGSGYSFSFHWIAWVRQMPGKGLEWMGIIHPGASDTRYSPSFQGQVTISADNSNSATYLQWSSLKASDTAMYFCARQRELDYFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 13 - protein, 236 aa, Homo sapiens
MDMRVPAQLLGLLLLWLRGARCDIQMTQSPSSLSASVGDRVTITCKASQDINKYVAWYQQKPGKAPKLLIYYTSWLQPGVPSRFSGSGSGTDFTFTISSLQPEDIATYYCLQYDNLLYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 14 - protein, 467 aa, Homo sapiens
MDMRVPAQLLGLLLLWLRGARCEVQLVESGGGLVQPGGSLRLSCAASGFTFSRYWMNWVRQAPGKGLEWVAQIRLKSDNYATHYAESVKGRFTISRDNAKNSLYLQMNSLRAEDTAVYYCTEGLDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - protein, 236 aa, Homo sapiens
MDMRVLAQLLGLLLLCFPGARCDIQMTQSPSSLSASVGDRVTITCRASQGISNWLAWYQQKPEKAPKSLIYAASSLQSGVPSRFSGSGSGTDFTLTISSLQPEDFATYYCQQYDSYPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 16 - protein, 466 aa, Homo sapiens
MELGLNWVFLVAILEGVHCEVQLVESGGGLVQPGGSLRLSCAASGFTFSSYWMSWVRQAPGKGLEWVAYIKQDGNEKYYVDSVKGRFTISRDNAKNSLYLQMNSLRAEDTAVYYCAREGILWFGDLPTFWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 17 - protein, 241 aa, Homo sapiens
MDMRLPAQLLGLLMLWVPGSSGDVLMTQSPLSLPVTLGQPASISCRSSQSIVHSNGNTYLEWYLQRPGQSPKLLIYKVSNRFSGVPDRFSGSGSGTDFTLKISRVEAEDVGVYYCFQGSHVPLTFGAGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 18 - protein, 470 aa, Homo sapiens
MDTLCSTLLLLTIPSWVLSQVTLKESGPALVKPTQTLTLTCTFSGFSLRTSGMGVGWIRQPPGKALEWLAHIWWDDDKSYNPSLKSQLTISKDTSKNQVVLTMTNMDPVDTATYYCARRNYYYDDYFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 19 - protein, 235 aa, Homo sapiens
METPAQLLFLLLLWLPDTTGEIVLTQSPGTLSLSPGERATLSCRASQSVSSSYFAWYQQKPGQAPRLLIYGTSSRATGIPDRFSGSGSGTDFTLTVSRLEPEDFAVYYCQQYDRSPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 20 - protein, 463 aa, Homo sapiens
MDWTWRILFLVAAATGAHSQVQLVQSGAAVKKPGASVKVSCKASGYTFTGYYIHWVRQAPGQGLEWMGWINPNSGGTNYAQKFQGRVTMTRDTSISTASMELSRLRSDDTAVYFCARDRWLDAFDIWGQGTMVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 21 - protein, 235 aa, Homo sapiens
METPAQLLFLLLLWLPDTTGEIVLTQSPGTLSLSPGERATLSCRASQSVSSGYLTWYQQKPGQAPRLLIYGASSRATGIPDRFSGSGSGTDFTLTISRLEPEDFAVYYCQQYGNSLSRFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 22 - protein, 474 aa, Homo sapiens
MDMRVPAQLLGLLLLWLRGARCQVQLVESGGGVVQPGRSLRLSCAASGFTFSSYGMHWVRQAPGKGLEWVAVIWYDGSNKYYADSVKGRFIISRDKSKNTLYLQMNSLRAEDTAVYYCARAGGIAAAGLYYYYGMDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 23 - protein, 236 aa, Homo sapiens
MDMRVPAQLLGLLLLWLRGARCSSELTQDPTVSVALGQTVKITCQGDSLRSFYASWYQQKPGQAPVLVFYGKNNRPSGIPDRFSGSSSGNTASLTITGAQAEDEADYYCNSRDSSVYHLVLGGGTKLTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 24 - protein, 478 aa, Homo sapiens
MDMRVPAQLLGLLLLWLRGARCQVQLVQSGAEVKKPGASVKVSCKASGYTFTGYYMHWVRQAPGQGLEWMGWINPNSGGTNYAQKFQGRVTMTRDTSISTAYMELSRLRSDDTAVYFCARDQMSIIMLRGVFPPYYYGMDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 25 - protein, 241 aa, Homo sapiens
MDMRVPAQLLGLLLLWLRGARCDIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 26 - protein, 475 aa, Homo sapiens
MDMRVPAQLLGLLLLWLRGARCEVQLVESGGGLVQPGGSLRLSCAASGFTFSSYWMSWVRQAPGKGLEWVASIKQDGSEKYYVDSVKGRFTISRDNARNSLYLQMNSLRAEDTAVYYCARDLVLMVYDIDYYYYGMDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 27 - protein, 241 aa, Homo sapiens
MDMRVPAQLLGLLLLWLRGARCDIVMTQSPLSLPVTPGEPASISCRSSQSLLHSNGYNYLDWYLQKPGQSPQLLIYLGSNRASGVPDRFSGSGSGTHLTLKISRVEAEDVGVYYCMQTLQTPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 28 - protein, 475 aa, Homo sapiens
MDMRVPAQLLGLLLLWLRGARCEVQLVESGGGLVQPGGSLRLSCAASGFTFSNYWMSWVRQAPGKGLEWVASIKQDGSEKYYVDSVKGRFAISRDNAKNSLFLQMNSLRAEDTAVYYCARDLVLMVYDIDYYYYGMDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 29 - protein, 235 aa, Homo sapiens
MTCSPLLLTLLIHCTGSWAQSVLTQPPSVSAAPGQKVTISCSGSSSNIGNNYVSWYQQLPGTAPKLLIYDNNKRPSGIPDRFSGSKSGTSTTLGITGLQTGDEADYYCGTWDSRLSAVVFGGGTKLTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 30 - protein, 475 aa, Homo sapiens
MEFGLSWVFLVALLRGVQCQVQLVESGGGVVQPGRSLRLSCAASGFTFSSFGMHWVRQAPGKGLEWVAVISFDGSIKYSVDSVKGRFTISRDNSKNTLFLQMNSLRAEDTAVYYCARDRLNYYDSSGYYHYKYYGMAVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 31 - protein, 234 aa, Homo sapiens
MDMRVPAQLLGLLLLWLRGARCYELTQPPSVSVSPGQTATITCSGDKLGERYASWYQQRPGQSPVLVIYQDIKRPSGIPERFSGSNSGNTATLTISGTQAMDEADYFCQAWYSSTNVLFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 32 - protein, 466 aa, Homo sapiens
MEFGLSWVFLVALLRGVQCQVQLVESGGGVVQPGRSLRLSCAASGFTFSSYGMHWVRQAPGKGLEWVAVIWYAESNKYYADSVKGRFTISRDNSKNTLYLQMNSLRAEDTAVYYCARAQEGIAPDAFDIWGQGTMVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 33 - protein, 234 aa, Homo sapiens
MGSTAILGLLLAVLQGGRAEIVLTQSPGTLSLSPGERATLSCRASQSVSSSYLAWHQQKPGQAPRLLIYGASSRATGIPDRFSGSGSGTDFTLTISRLEPEDFAVYYCQQYGSSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 34 - protein, 463 aa, Homo sapiens
MGSTAILGLLLAVLQGGRAEVQLLESGGGLVQPGGSLRLSCAASGFTFSTYVMSWVRQAPGKGLEWVSSISGSGLGSYYADSVKGRFTISRDNSKNTLYLQMNSLRAEDTAVYYCAKEAHRGPFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 35 - protein, 239 aa, Homo sapiens
MRLLAQLLGLLMLWVPGSSGDIVMTQTPLSSPVTLGQPASISCRSSQSLVHSDGNTYLSWLQQRPGQPPRLLIYKKFNRFSGVPDRFSGSGAGTDFTLKISRVEAEDVGVYYCMQATQIPLTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 36 - protein, 472 aa, Homo sapiens
MEFGLSWVFLVALLRGVQCQVQLVESGGGVVQPGRSLRLSCAASGFTFSFYGMHWVRQAPGKGLEWVAVIWYDGSNKYYADSVKGRFTISRDNSKNTLYLQMNSLRAEDTAVYYCARGGYDYVWGSYRRNSDFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 37 - protein, 235 aa, Homo sapiens
MEAPAQLLFLLLLWLPDTTGEIVMTQSPATLSVSPGERATLSCRASQSVDSNLAWYRQKPGQAPRLLIYGASTRATGIPARFSGSGSGTEFTLTISSLQSEDFAVYYCQQYINWPPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 38 - protein, 465 aa, Homo sapiens
MKHLWFFLLLVAAPRWVLSQVQLQESGPGLVKPSETLSLTCTVSGGSISIYYWSWIRQPPGKGLEWIGYVYYSGSTNYNPSLKSRVTISVDTSKNQFSLKLNSVTAADTAVYYCARGGYDFWSGYFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 39 - protein, 240 aa, Homo sapiens
MVLQTQVFISLLLWISGAYGDIVLTQSPDSLAVSLGERATINCKSSQSILYSSSNENFLTWYQQKPGQPPKLLIYWASTRESGVPDRFSGSGSGTDFTLTISSLQPEDVAVYYCQQYFSVFRTFGQGTRVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 40 - protein, 465 aa, Homo sapiens
MDWTWRILFLVAAATGAHSQVQLVQSGAEVKKPGASVKVSCKASGYTFTGYYIHWVRQAPGQGLEWMGWINPNSGGTNSAQKFRGRVTMTRDTSISTAYMELSRLRSDDTAVYYCAREGGYSYGYFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 41 - protein, 236 aa, Homo sapiens
MDMRVPAQLLGLLLLWFPGARCDIQMTQSPSSLSASVGDRVTITCRASQGIRNDLGWYQQKPGKAPKRLIYAASSLQSGVPSRFSGSGSGTEFTLTISSVQPEDFVTYYCLQHNSNPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 42 - protein, 473 aa, Homo sapiens
MEFGLSWVFLVALLRGVQCQVQLVESGGGVVQPGRSLRLSCAASGFTFSSYGMHWVRQAPGKGLEWVAVMWYDGSNKDYVDSVKGRFTISRDNSKNTLYLQMNRLRAEDTAVYYCAREKDHYDILTGYNYYYGLDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 43 - protein, 235 aa, Homo sapiens
METPAQLLFLLLLWLPDTTGEIVLTQSPGTLSLSPGERATLSCRASQYISNTYLAWFQQKPGQAPRLLIYGAATRATGIPDRFSGSGSGTDFTFTISRLEPEDFAVYYCQQYGSSPWTFGQGTTVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 44 - protein, 463 aa, Homo sapiens
MDWTWRILFLVAAATGAHSQVQLVQSGAEVKKPGASVKVSCKASGYTFTGYYMHWVRQAPGQGLEWMGWINPNSGGTNYAQRFRGRVTMTRDTSISTAYMELSRLRSDDTAVYYCARAPYDWTFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 45 - protein, 236 aa, Homo sapiens
MDMRVPAQLLGLLLLWLRGARCDIQMTQSPSSVSASVGDRVTITCRASQGISNWLAWYQQKPGTAPKLLIYAASSLQSGVPSRFSGSGSGTDFTLTISSLQPEDFATYYCQQANSFPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 46 - protein, 469 aa, Homo sapiens
MDMRVPAQLLGLLLLWLRGARCQVQLVQSGTEVKKPGASMKVSCKASGYTFTSYYMHWVRQAPGQGLEWMGIINPSGDSTSYAQKFQGRVTMTRDTSTNTVYMELSSLRSEDTAMYYCARDVEVRGISHFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 47 - protein, 238 aa, Homo sapiens
MGWSCIILFLVATATGVHSDIQLTQSPSSLSASVGDRVTMSCKSSQSVLYSANHKNYLAWYQQKPGKAPKLLIYWASTRESGVPSRFSGSGSGTDFTFTISSLQPEDIATYYCHQYLSSWTFGGGTKVQIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 48 - protein, 465 aa, Homo sapiens
MGWSCIILFLVATATGVHSQVQLVQSGAEVKKPGSSVKVSCKASGYTFTSYWLHWVRQAPGQGLEWIGYINPRNDYTEYNQNFKDKATITADESTNTAYMELSSLRSEDTAFYFCARRDITTFYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 49 - protein, 235 aa, Homo sapiens
METPAQLLFLLLLWLPDTTGEIVLTQSPGTLSLSPGERATLSCRASQSVSSSYLAWYQQKPGQAPRLLIYGASSRATGIPDRFSGSGSGTDFTLTISRLEPEDFAVYYCQRSGGSSFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 50 - protein, 466 aa, Homo sapiens
MGSTAILALLLAVLQGVCAEVQLVQSGAEVKKPGESLKISCKGSGYNFTSYWIGWVRQMPGKGLELMGIIYPGDSDTRYSPSFQGQVTISADKSISTAYLQWSSLKASDTAMYYCGSGSYFYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 51 - protein, 241 aa, Homo sapiens
MDMRVPAQLLGLLLLWLRGARCDIVMTQSPLSLPVTPGEPASISCRSSQSLLHSHGYNYLDWYLQKPGQSPQLLIYLGSNRASGVPDRFSGSGSGTDFTLKISRVEAEDVGVYYCMQGTHWPPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 52 - protein, 470 aa, Homo sapiens
MDMRVPAQLLGLLLLWLRGARCEVQLVQSGGGVVQPGRSLRLSCAASGFTFSSYGMHWVRQAPGKGLEWVSYISSSGSTIYYADSVKGRFTISRDNAKNSLYLQMNSLRAEDTAVYYCARDLLDYDLLTGYGYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 53 - protein, 235 aa, Homo sapiens
METPAQLLFLLLLWLPDTTGEIVLTQSPGTLSLSPGERATLSCRASQGISRSYLAWYQQKPGQAPSLLIYGASSRATGIPDRFSGSGSGTDFTLTISRLEPEDFAVYYCQQFGSSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 54 - protein, 471 aa, Homo sapiens
MKHLWFFLLLVAAPRWVLSQVQLQESGPGLVKPSQTLSLTCTVSGGSISSGDYFWSWIRQLPGKGLEWIGHIHNSGTTYYNPSLKSRVTISVDTSKKQFSLRLSSVTAADTAVYYCARDRGGDYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 55 - protein, 236 aa, Homo sapiens
MDMRVPAQLLGLLLLWLSGARCDIQMTQSPSSLSASVGDRVTITCQASQDISNYLNWYQQKPGKAPKLLIYDASNLETGVPSRFSGSGSGTDFTFTISSLQPEDIATYFCQHFDHLPLAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 56 - protein, 464 aa, Homo sapiens
MKHLWFFLLLVAAPRWVLSQVQLQESGPGLVKPSETLSLTCTVSGGSVSSGDYYWTWIRQSPGKGLEWIGHIYYSGNTNYNPSLKSRLTISIDTSKTQFSLKLSSVTAADTAIYYCVRDRVTGAFDIWGQGTMVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 57 - protein, 236 aa, Homo sapiens
METPAQLLFLLLLWLPDTASEIVLTQSPGTLSLSPGERATLSCRASQSVSNSYLAWYQQKPGQAPRLLIYGASSRAPGIPDRFSGSGSGTDFTLTISRLEPEDFAVYYCQQYDHSAGWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 58 - protein, 460 aa, Homo sapiens
MGSTAILGLLLAVLQGVASEVQLVESGGGLVQPGGSLRLSCAASGFTFSRNAMFWVRQAPGKGLEWVSGIGTGGATSYADSVKGRFTISRDNAKNSLYLQMNSLRAEDTAVYYCARGRYYFPWWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 59 - protein, 234 aa, Homo sapiens
METPAQLLFLLLLWLPDTTGEIVLTQSPGTLSLSPGERATLSCRASQSVSSSYLAWYQQKPGQAPRLLIYGASRRATGIPDRFSGSGSGTDFTLTISRLEPEDFAVYYCQRYGSSHTFGQGTKLEISRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 60 - protein, 467 aa, Homo sapiens
MGSTAILALLLAVLQGVCAEVQLVQSGAEVKKPGESLKISCKVSGYFFTTYWIGWVRQMPGKGLEYMGIIYPGDSDTRYSPSFQGQVTISADKSISTAYLQWSSLKASDTAMYYCARGGNWNCFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 61 - protein, 241 aa, Homo sapiens
MDMRVPAQLLGLLLLWLRGARCDVVMTQSPLSLPVTPGEPASISCRSSQSLLHSNGYNYLDWYLQKPGQSPQLLIYLGSNRASGVPDRFSGSGSGTDFTLKISRVEAEDVGVYYCMQGTHWPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 62 - protein, 471 aa, Homo sapiens
MDMRVPAQLLGLLLLWLRGARCQVQLQESGPGLVKPSGTLSLTCAVSGGSISSSNWWSWVRQPPGKGLEWIGEIYHSGSTNYNPSLKSRVTISVDKSKNQFSLKLSSVTAADTAVYYCARWTGRTDAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 63 - protein, 234 aa, Homo sapiens
MEAPAQLLFLLLLWLPDTTGEIVMTQSPATLSVSPGERATLSCRASQSVSSNLAWFQQKPGQAPRPLIYDASTRATGVPARFSGSGSGTDFTLTISSLQSEDFAVYYCQQYDNWPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 64 - protein, 461 aa, Homo sapiens
MEWTWRVLFLVAAATGAHSQVQLVQSGAEVKKPGASVKVSCKASGYTFTRYGISWVRQAPGQGLEWMGWISTYSGNTNYAQKLQGRVTMTTDTSTSTAYMELRSLRSDDTAVYYCARRQLYFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 65 - protein, 239 aa, Homo sapiens
MRLPAQLLGLLMLWIPGSSADIVMTQTPLSLSVTPGQPASISCKSGQSLLHSDGKTYLYWYLQKPGQPPQFLIYEVSNRFSRVPDRFSGSGSGTDFTLRISRVEAEDVGIYYCMQSIQLPWTFGQGTQVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 66 - protein, 459 aa, Homo sapiens
MEFGLSWVFLVALLRGVQCQVQLVESGGGVVQPGRSLRLSCAASGFTFSGYGMHWVRQAPGKGLEWVAVISYDGNDKYYADSVKGRFTISRDNAKNTLYLQMNSLRAEDTAVYYCARELRVLWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 67 - protein, 235 aa, Homo sapiens
MDMRVPAQLLGLLLLWLRGARCDIQMTQSPSSLSASVGDRVTITCRASQDISSYLNWYQQKPGKAPKLLIYSTSRLNSGVPSRFSGSGSGTDFTLTISSLQPEDFATYYCQQDIKHPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 68 - protein, 465 aa, Homo sapiens
MDWTWRILFLVAAATGAHSEVQLVQSGAEVKKPGSSVKVSCKASGFTFTDYIMHWVRQAPGQGLEWMGYINPYNDDTEYNEKFKGRVTITADKSTSTAYMELSSLRSEDTAVYYCARSIYYYDAPFAYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 69 - protein, 233 aa, Homo sapiens
MGSTAILGLLLAVLQGGRASYVLTQPPSVSVAPGQTARITCGGNNLGSKSVHWYQQKPGQAPVLVVYDDSDRPSWIPERFSGSNSGNTATLTISRGEAGDEADYYCQVWDSSSDHVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 70 - protein, 467 aa, Homo sapiens
MGSTAILGLLLAVLQGGRAQMQLVESGGGVVQPGRSLRLSCAASGFTFRTYGMHWVRQAPGKGLEWVAVIWYDGSNKHYADSVKGRFTITRDNSKNTLNLQMNSLRAEDTAVYYCARAPQWELVHEAFDIWGQGTMVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 71 - protein, 234 aa, Homo sapiens
MGVPTHLLGLLLLWITHAICDIRMTQSPASLSASLGETVNIECLASEDIYSDLAWYQQKPGKSPQLLIYNANSLQNGVPSRFSGSGSGTQYSLKINSLQSEDVATYFCQQYNNYPPTFGGGTKLELKRADAAPTVSIFPPSTEQLATGGASVVCLMNNFYPRDISVKWKIDGTERRDGVLDSVTDQDSKDSTYSMSSTLSLTKADYESHNLYTCEVVHKTSSSPVVKSFNRNEC

SEQ ID NO: 72 - protein, 465 aa, Homo sapiens
MDIRLSLAFLVLFIKGVQCEVQLVESGGGLVQPANSLKLSCAASGFTFSDYAMAWVRQSPKKGLEWVATIIYDGSSTYYRDSVKGRFTISRDNAKSTLYLQMDSLRSEDTATYYCATGLGIATDYFDYWGQGVLVTVSSAETTAPSVYPLAPGTALKSNSMVTLGCLVKGYFPEPVTVTWNSGALSSGVHTFPAVLQSGLYTLTSSVTVPSSTWPSQTVTCNVAHPASSTKVDKKIVPRNCGGDCKPCICTGSEVSSVFIFPPKPKDVLTITLTPKVTCVVVDISQDDPEVHFSWFVDDVEVHTAQTRPPEEQFNSTFRSVSELPILHQDWLNGRTFRCKVTSAAFPSPIEKTISKPEGRTQVPHVYTMSPTKEEMTQNEVSITCMVKGFYPPDIYVEWQMNGQPQENYKNTPPTMDTDGSYFLYSKLNVKKEKWQQGNTFTCSVLHEGLHNHHTEKSLSHSPGK

SEQ ID NO: 73 - protein, 237 aa, Homo sapiens
MDMRVPAQLLGLLLLWLRGARCESALTQPASVSGSPGQSITISCTGTSSDVGGYNSVSWYQQHPGKAPKLMIYEVSNRPSGVSNRFSGSKSGNTASLTISGLQAEDEADYYCNSYTSTSMVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 74 - protein, 460 aa, Homo sapiens
MDWTWRILFLVAAATGVHSEVQLVQSGAEVKKPGASVKVSCKASGYTLTSYGISWVRQAPGQGLEWMGWVSFYNGNTNYAQKLQGRGTMTTDPSTSTAYMELRSLRSDDTAVYYCARGYGMDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 75 - protein, 234 aa, Homo sapiens
METPAQLLFLLLLWLPDTTGEIVMTQSPATLSVSPGERATLSCRASQSVSSNLAWYQQKPGQAPRLLIYGAATRATGIPARVSGSGSGTEFTLTISSLQSEDFAVYYCQQYNNWPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 76 - protein, 472 aa, Homo sapiens
MDMRVPAQLLGLLLLWLRGARCQVQLVESGGGVVQPGRSLRLSCAASGFTFSNYGMHWVRQAPGEGLEWVAAIWFDASDKYYADAVKGRFTISRDNSKNTLYLQMNSLRAEDTAVYYCARDQAIFGVVPDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 77 - protein, 231 aa, Homo sapiens
MAWIPLFLGVLAYCTGSVASYEVTQAPSVSVSPGQTASITCSGDKLGDKYACWYQQKPGQSPVLVIYQDSKRPSGIPERFSGSNSGNTATLTISGTQAMDEADYYCQAWDSSTAVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 78 - protein, 467 aa, Homo sapiens
MDWTWSILFLVAAATGAHSQVQLVQSGAEVKKPGASVKVSCKASGYTFTSYGLSWVRQAPGQGLEWMGWIIPYNGNTNSAQKLQGRVTMTTDTSTSTAYMELRSLRSDDTAVYFCARDRDYGVNYDAFDIWGQGTMVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 79 - protein, 235 aa, Homo sapiens
MAWAPLLLTLLAHCTGSWANFMLTQPHSVSESPGKTVAISCTRNSGSIASNSVQWYQQRPGSSPTTVIFEDNQRPSGVPDRFSGSIDSSSNSASLTISGLKTEDEADYYCQSYDSNNWVFGGGTKLTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 80 - protein, 470 aa, Homo sapiens
MEFGLSWVFLVALLRGVQCQVQLVESGGGVVQPGRSLRLSCAASGFTFSSYVMHWVRQAPGKGLEWVAVIWYDGSNKYYADSVKGRFTISRDNSKNTLYLQMNSLRAEDTAVYYCAREGYDYGEDYYYYGMDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 81 - protein, 236 aa, Homo sapiens
MDMRVPAQLLGLLLLWLRGARCDIQMTQSPSSLSASVGDRVTITCRASQDISNYLNWYQQKPGKAPKLLIYYTSRLLSGVPSRFSGSGSGTDFTLTISSLQPEDFATYYCQQGDTLPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 82 - protein, 468 aa, Homo sapiens
MDWTWRILFLVAAATGAHSEVQLVQSGAEVKKPGASVKVSCKASGYTFTDYNMHWVRQAPGQGLEWMGEINPNSGGAGYNQKFKGRVTMTTDTSTSTAYMELRSLRSDDTAVYYCARLGYDDIYDDWYFDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 83 - protein, 240 aa, Homo sapiens
MVLQTQVFISLLLWISGAYGDIVMTQSPDSLAVSLGERATINCKSSQSVLDSSDNKNYLAWYQQKPGQPPKLLIYWASNRESGVPDRFSGSGSGTDFTLTISSLQAEDVAVYYCQQYYSDPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 84 - protein, 465 aa, Homo sapiens
MDWTWSILFLVAAPTGAHSQVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARESWFGEVFFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 85 - protein, 236 aa, Homo sapiens
MDMRVPAQLLGLLLLWLRGARCDIQMTQSPSSVSASVGDRVTITCRASQGISSWLAWYQQKPGKAPKLLIYGASNLESGVPSRFSGSGSGTDFTLTISSLQPEDFANYYCQQANSFPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 86 - protein, 466 aa, Homo sapiens
MDMRVPAQLLGLLLLWLRGARCQVQLVQSGAEVKKPGASVKVSCKVSGYTLSDLSIHWVRQAPGKGLEWMGGFDPQDGETIYAQKFQGRVTMTEDTSTDTAYMELSSLKSEDTAVYYCATGSSSSWFDPWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 87 - protein, 236 aa, Artificial sequence
  Synthetic
MDMRVPAQLLGLLLLWLRGARCDIQMTQSPSSVSASVGDRVTITCRASQGISNWLAWYQQKPGKAPKLLIFAASSLQSGVPSRFSGSGSGTDFTLTISSLQPEDFATYYCQQAESFPHTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 88 - protein, 472 aa, Artificial sequence
  Synthetic
MDMRVPAQLLGLLLLWLRGARCEVQLLESGGGLVQPGGSLRLSCAASGFTFSSYDMSWVRQAPGKGLEWVSLISGGGSQTYYAESVKGRFTISRDNSKNTLYLQMNSLRAEDTAVYFCASPSGHYFYAMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2023/0035363 A1
(43) Pub. Date: Feb. 2, 2023

(54) IN VIVO REVERSIBILITY OF HIGH MOLECULAR WEIGHT SPECIES

(71) Applicant: AMGEN INC., Thousand Oaks, CA (US)

(72) Inventors: Dong XIANG, Thousand Oaks, CA (US); Qingchun ZHANG, Oak Park, CA (US); Marisa JOUBERT, Newbury Park, CA (US); Trent MUNRO, Kenmore (AU); Ronandro DE GUZMAN, Camarillo, CA (US)

(21) Appl. No.: 17/436,218

(22) PCT Filed: Mar. 4, 2020
(86) PCT No.: PCT/US2020/020956

§ 371 Date: Sep. 3, 2021

Related U.S. Application Data
(60) Provisional application No. 62/813,529, filed on Mar. 4, 2019.
(60) Provisional application No. 62/944,758, filed on Dec. 6, 2019.

(51) Int. Cl. G01N 33/68; G01N 30/14; G01N 21/64

(52) U.S. Cl. CPC G01N 33/6845; G01N 30/14; G01N 21/6428; G01N 2021/6439; G01N 2030/027

(57) ABSTRACT

Provided herein are in vitro methods of assaying an in vivo level of high molecular weight (HMW) species of a therapeutic protein. In exemplary embodiments, the method comprises (a) incubating a mixture comprising (i) a sample comprising the therapeutic protein and (ii) serum, or a depleted fraction thereof; and (b) assaying the level of HMW species of the therapeutic protein present in the mixture at one or more time points after step (a). Also methods of determining the in vivo reversibility of HMW species of a therapeutic protein are provided herein. In exemplary instances, the method comprises (A) assaying the in vivo level of high molecular weight (HMW) species of a therapeutic protein according to a presently disclosed in vitro method, and (B) comparing the level(s) of HMW species present in the mixture to the level of HMW species present in the sample prior to the incubating step.

### CROSS REFERENCE TO RELATED APPLICATIONS

The benefit under 35 U.S.C. § 119(e) of U.S. Provisional Application No. 62/813,529, filed Mar. 4, 2019, and U.S. Provisional Application No. 62/944,758, filed Dec. 6, 2019, is hereby claimed and the entire contents thereof are incorporated herein by reference.

### INCORPORATION BY REFERENCE OF MATERIAL SUBMITTED ELECTRONICALLY

Incorporated by reference in its entirety is a computer-readable nucleotide/amino acid sequence listing submitted concurrently herewith and identified as follows: 270,234 byte ASCII (Text) file named “53990_Seqlisting.txt”; created on Mar. 4, 2020.

### BACKGROUND

The native structures of proteins are designed to adapt to changes within the protein's environment. Although structure flexibility is needed for the biological function of proteins, it also presents many challenges during the development of therapeutic proteins for pharmaceutical applications. Chemical modifications of amino acid residues, conformational changes, aggregation, and precipitation, which are associated with the loss of biological activity and the immunogenicity of proteins, increase the difficulty of developing certain proteins as therapeutics. During each of the many steps that lead up to administration of a therapeutic protein (e.g., production, harvest, purification, formulation, storage and delivery), these proteins are susceptible to undergoing modifications and change in structure, and as a result, varying species are formed. The formation of High Molecule Weight (HMW) species of therapeutic proteins represent one type of modification that can occur during these pre-administration steps. HMW species remain a concern for the biopharmaceutical industry from the standpoint of safety and efficacy, because HMW species can exhibit a reduced therapeutic efficacy and can lead to undesirable immunological responses once administered to patients. Also, given that the individual components (therapeutic proteins) of HMW species are joined together via non-covalent bonds and the in vivo environment is substantially different from the in vitro context (e.g., a packaged formulation of the therapeutic protein), the amount and type of HMW species can change once administered to the patient. It is therefore desirable to determine the amount and type of HMW species of therapeutic proteins not only before administration to the patient, but also after administration-in an in vivo context. While many researchers study the phenomenon of HMW species formation in in vitro contexts (e.g., in tubes where the therapeutic proteins are in buffers isolated from other proteins), such studies are not predictive of the fate of the HMW species following administration to a patient. Few investigators have aimed to analyze the association and dissociation of HMW species of therapeutic proteins in a more in vivo context in an in vitro assay (e.g., in the presence of blood proteins and/or blood cells) due to the limitations of the techniques used to measure HMW species in such contexts. For example, proteins found in the in vivo context mask the signals of the therapeutic proteins and HMW species thereof.

Accordingly, being able to predict the amount and type of HMW species of therapeutic proteins once inside the body of the patient is highly desirable, and thus there is a need for in vitro methods of assaying the level of HMW species of a therapeutic protein in relevant in vivo contexts.

### SUMMARY

Described herein for the first time are in vitro methods of assaying the level of HMW species of a therapeutic protein while the therapeutic proteins (and the HMW species thereof) are present in an environment that mimics the post-administration, in vivo context. The data presented herein support that the presently disclosed methods are capable of successfully monitoring the amount and type of HMW species over time in an engineered in vivo context, despite the presence of serum proteins which ordinarily mask the signal of the therapeutic protein and HMW species thereof. The presently disclosed methods can advantageously determine the reversibility of HMW species formation of a therapeutic protein in an in vivo context, which characteristic or parameter is referenced herein as the in vivo reversibility of HMW species of a therapeutic protein.

Accordingly, the present disclosure provides an in vitro method of assaying an in vivo level of high molecular weight (HMW) species of a therapeutic protein. In a first aspect in exemplary embodiments, the method comprises (A) incubating a mixture comprising (i) a sample comprising the therapeutic protein and (ii) serum, or a depleted fraction thereof; and (B) assaying the level of HMW species of the therapeutic protein present in the mixture at one or more time points after step (a). Alternatively or additionally, the level of HMW species of the therapeutic protein in the mixture is assayed by size-exclusion chromatography (SEC).

Also provided, in a second aspect, are methods of determining the in vivo reversibility of HMW species of a therapeutic protein. In exemplary embodiments, the method comprises (A) assaying the in vivo level of high molecular weight (HMW) species of a therapeutic protein according to the first aspect, wherein (i) the method further comprises assaying the level of HMW species present in the sample prior to the incubating step (step (A)) or (ii) the level of HMW species present in the sample prior to the incubating step (step (A)) is known and (B) comparing the level(s) of HMW species present in the mixture to the level of HMW species present in the sample prior to the incubating step (step (A)).

In exemplary embodiments, the method of determining the in vivo reversibility of HMW species of a therapeutic protein comprises: incubating a mixture comprising a sample comprising the therapeutic protein and a depleted serum, wherein the depleted fraction of serum is a fraction depleted of molecules having a pre-selected molecular weight range, optionally, wherein the pre-selected molecular weight range is about 30 kDa to about 300 kDa or higher, optionally, wherein the depleted fraction is obtained through size-based filtration; assaying the level of HMW species of the therapeutic protein present in the mixture at one or more time points after step (a) by SEC; comparing the level(s) of the HMW species present in the mixture as assayed in step (b) to the level of the HMW species present in the sample prior to step (a); and calculating the percentage of in vivo reversibility of the HMW species of the therapeutic protein.

In exemplary embodiments, the method of determining the in vivo reversibility of HMW species of a therapeutic protein comprises: incubating a mixture comprising a sample comprising the therapeutic protein and a depleted serum, wherein the depleted serum is an IgG-depleted serum fraction, optionally, obtained by removing IgG from serum by Protein A affinity chromatography; separating components of the mixture by affinity chromatography with a capture molecule to obtain a fraction comprising the therapeutic protein and HMW species thereof; assaying the level of HMW species of the therapeutic protein present in the fraction by SEC, comparing the level(s) of the HMW species present in the fraction as assayed in step (c) to the level of the HMW species present in the sample prior to step (a); and calculating the percentage of in vivo reversibility of the HMW species of the therapeutic protein.

In exemplary embodiments, the method of determining the in vivo reversibility of HMW species of a therapeutic protein comprises: incubating a mixture comprising a sample comprising the therapeutic protein with whole serum, wherein the therapeutic protein comprises a fluorescent label; diluting the mixture; assaying the level of HMW species of the therapeutic protein present in the mixture at one or more time points after step (a) by SEC, comparing the level(s) of the HMW species present in the mixture as assayed in step (c) to the level of the HMW species present in the sample prior to step (a); and calculating the percentage of in vivo reversibility of the HMW species of the therapeutic protein.

In exemplary embodiments, the method of determining the in vivo reversibility of HMW species of a therapeutic protein comprises: incubating a mixture comprising a sample comprising the therapeutic protein and whole serum; separating components of the mixture by affinity chromatography with a capture molecule to obtain a fraction comprising the therapeutic protein and HMW species thereof; assaying the level of HMW species of the therapeutic protein present in the fraction by SEC, comparing the level(s) of the HMW species present in the fraction as assayed in step (c) to the level of the HMW species present in the sample prior to step (a); and calculating the percentage of in vivo reversibility of the HMW species of the therapeutic protein.

### BRIEF DESCRIPTION OF THE DRAWINGS

FIG. 1 is a schematic of four exemplary methods of determining in vivo reversibility of HMW species of a therapeutic protein.

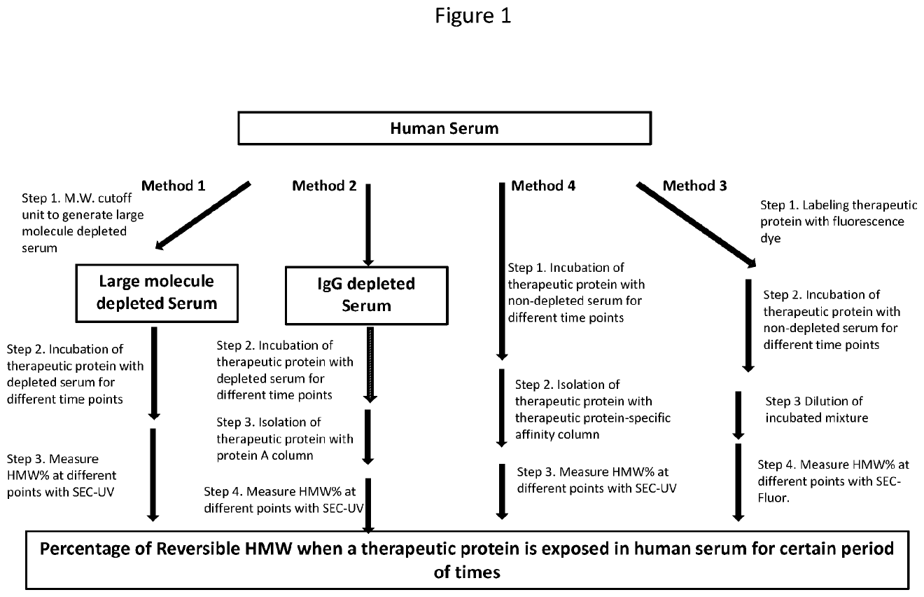

FIG. 2 is an overlay of SEC chromatograms of aliquots taken at different time points during the incubation period. The mixture comprised a diluted sample of TP2 (10% HMW) in depleted human serum. Peaks for HMW species (HMW) and monomeric therapeutic protein (Monomer) are shown.

FIG. 3 is an overlay of SEC chromatograms of aliquots of the mixture taken at different time points during the incubation period. The mixture comprised a diluted sample of TP2 (5% HMW) in depleted human serum. Peaks for HMW species (HMW) and monomeric therapeutic protein (Monomer) are shown.

FIG. 4 is a set of SEC-HPLC spectra showing peaks representative of the therapeutic protein monomer, HMW species, and a co-eluting serum component (“post-peak”).

FIG. 5 is a series of SEC-HPLC spectra obtained using different elution buffers.

FIG. 6 is a series of SEC-HPLC spectra obtained. during the initial TP1 stability evaluation in potential elution buffers and wash buffers.

### DETAILED DESCRIPTION

The present disclosure provides an in vitro method of assaying an in vivo level of high molecular weight (HMW) species of a therapeutic protein. In exemplary embodiments, the method comprises (A) incubating a mixture comprising (i) a sample comprising the therapeutic protein and (ii) serum, or a depleted fraction thereof; and (B) assaying the level of HMW species of the therapeutic protein present in the mixture at one or more time points after step (a). In exemplary aspects, the level of HMW species of the therapeutic protein in the mixture is assayed by size-exclusion chromatography (SEC).

By “assaying” is meant “testing” or “analyzing” or “determining”. In some aspects, “assaying” means “measuring”. The level that is assayed or determined by the presently disclosed methods can be a relative measurement, e.g., a determination that the level is higher or lower or the same as a reference level. In exemplary aspects, the reference level is the level of HMW species prior to being mixed with serum or a depleted fraction thereof, or the level of HMW species in the formulation for administration to a subject. In such aspects, the method of the present disclosure assays the level of HMW species and can determine that the level of HMW species is higher or lower or the same as the level of HMW species prior to being mixed with serum or higher or lower or the same as the level of HMW species in the formulation for administration to a subject. The “assaying” in some aspects can yield a normalized measurement. For instance, the normalized measurement can be normalized to a reference protein, e.g., serum albumin. The “assaying” in certain instances yields an absolute measurement (e.g., neither normalized nor relative to a reference level).

The “in vivo level of high molecular weight (HMW) species of a therapeutic protein” assayed by the in vitro method of the present disclosure is, in exemplary instances, a predicted level of HMW species of the therapeutic protein based on placing the therapeutic protein (and HMW species thereof) in an in vivo-like context. In exemplary aspects, the “in vivo level of high molecular weight (HMW) species of a therapeutic protein” is a level that is useful for forecasting what happens in vivo to the HMW species of a therapeutic protein post-administration to a subject. In exemplary aspects of the presently disclosed methods of assaying an in vivo level of high molecular weight (HMW) species of a therapeutic protein, the method further comprises assaying the level of HMW species present in the sample prior to the incubating step (step (a)). In various instances, the level of HMW species present in the sample prior to the incubating step (step (a)) is known. Methods of assaying the level of HMW species present in the sample prior to the incubating step (step (a)) can be performed according to any known suitable technique. In some aspects, the level of HMW is assayed as described herein and comprises size exclusion chromatography (SEC)-high performance liquid chromatography (HPLC).

As used herein “HMW species” in reference to a therapeutic protein means a formed aggregate of two or more molecules (therapeutic proteins) linked by non-covalent bonds. HMW species include, but are not limited, to dimers (comprising 2 therapeutic proteins), trimers (comprising 3 therapeutic proteins), tetramers (comprising 4 therapeutic proteins), pentamers (comprising 5 therapeutic proteins), hexamers (comprising 6 therapeutic proteins), heptamers (comprising 7 therapeutic proteins), and octamers (comprising 8 therapeutic proteins), of a therapeutic protein. In exemplary aspects, a HMW species can be of higher order, e.g., can comprise more than 8 therapeutic proteins. For instance, the HMW species can be a enneamer (comprising 9 therapeutic proteins), decamer (comprising 10 therapeutic proteins), hendecamer (comprising 11 therapeutic proteins), dodecamer (comprising 12 therapeutic proteins), triadecamer (comprising 13 therapeutic proteins), quatrodecamer (comprising 14 therapeutic proteins), quindecamer (comprising 15 therapeutic proteins), sexdecamer (comprising 16 therapeutic proteins), septendecamer (comprising 17 therapeutic proteins), octodecamer (comprising 18 therapeutic proteins), or a novendecamer (comprising 19 therapeutic proteins). In various embodiments, the HMW species assayed by the presently disclosed methods can comprise one or more of dimers, trimers, tetramers, pentamers, hexamers, heptamers, and octamers, of the therapeutic protein.

In exemplary aspects, the size of the HMW species assayed by the presently disclosed methods is less than about 0.1 microns (100 nm). Optionally, the size of the HMW species is about 99 nm or less. In exemplary aspects, the size of the HMW species is greater than about 10 nm and less than about 99 nm. In exemplary aspects, the size of the HMW species is greater than about 15 nm and less than about 99 nm. In exemplary aspects, the size of the HMW species is about 15 nm to about 99 nm, about 20 nm to about 99 nm, about 30 nm to about 99 nm, about 40 nm to about 99 nm, about 50 nm to about 99 nm, about 60 nm to about 99 nm, about 70 nm to about 99 nm, about 80 nm to about 99 nm, about 90 nm to about 99 nm. In exemplary instances, the size of the HMW species is about 15 nm to about 90 nm, about 15 nm to about 80 nm, about 15 nm to about 70 nm, about 15 nm to about 60 nm, about 15 nm to about 50 nm, about 15 nm to about 40 nm, about 15 nm to about 30 nm, or about 15 nm to about 20 nm.

In various aspects, the size of the HMW species is less than about 15 nm. Optionally, the size of the HMW species is less than about 10 nm or less than about 5 nm.

In exemplary aspects, the method further comprises assaying the level of one or more of dimers, trimers, tetramers, pentamers, hexamers, heptamers, and octamers, of the therapeutic protein prior to step (a). In various instances, the level of one or more of dimers, trimers, tetramers, pentamers, hexamers, heptamers, and octamers, of the therapeutic protein present in the sample prior to step (a) is known. In exemplary aspects, the assaying step (step (b)) comprises assaying the level of each of dimers, trimers, tetramers, pentamers, hexamers, heptamers, or octamers, of the therapeutic protein.

As used herein, the term “therapeutic protein,” which is synonymous with “therapeutic polypeptide,” refers to any protein or polypeptide molecule, which can be naturally-occurring or non-naturally-occurring (e.g., engineered or synthetic), comprising at least one polypeptide chain which has or is intended to have therapeutic efficacy when administered to a subject for treatment of a disease or medical condition. When two therapeutic proteins have the same amino acid sequence, the two therapeutic proteins are considered as the same therapeutic protein.

In exemplary aspects, the therapeutic protein is a recombinant protein. By “recombinant protein” means any protein or polypeptide that results from the expression of recombinant DNA within living cells. The term “recombinant DNA” means any DNA molecule formed through genetic recombination (e.g., molecular cloning) of genetic material from multiple sources to create DNA molecules that are not found in any naturally-occurring genome. The multiple sources may be from a different molecule or from a different part of the same molecule. The recombinant DNA in some aspects encodes a naturally-occurring protein. In other aspects, the recombinant DNA encodes a protein that does not exist in nature (e.g., non-naturally-occurring).

In various aspects, the therapeutic protein is an antibody, antigen-binding fragment of an antibody, or an antibody protein product. In exemplary aspects, the therapeutic protein is a hormone, growth factor, cytokine, a lymphokine, a fusion protein, a cell-surface receptor, or any ligand thereof. Exemplary therapeutic proteins are known in the art and also described herein.

With regard to the presently disclosed methods of assaying an in vivo level of HMW species, the method comprises incubating a mixture, wherein the mixture comprises a sample comprising the therapeutic protein and serum, or a depleted fraction thereof. In exemplary aspects, the therapeutic protein is present in the mixture at a final concentration of about 10 μg/mL to about 300 μg/mL. In certain instances, the therapeutic protein is present in the mixture at a final concentration of, about 10 μg/mL to about 250 μg/mL, about 10 μg/mL to about 200 μg/mL, about 10 μg/mL to about 150 μg/mL, about 10 μg/mL to about 100 μg/mL, about 10 μg/mL to about 75 μg/mL, about 10 μg/mL to about 50 μg/mL, about 10 μg/mL to about 25 μg/mL, about 25 μg/mL to about 300 μg/mL, about 50 μg/mL to about 300 μg/mL, about 75 μg/mL to about 300 μg/mL, about 100 μg/mL to about 300 μg/mL, about 150 μg/mL to about 300 μg/mL, about 200 μg/mL to about 300 μg/mL, or about 250 μg/mL to about 300 μg/mL, including 50 μg/mL, 60 μg/mL, 70 μg/mL, 75 μg/mL, 80 μg/mL, 90 μg/mL, 100 μg/mL, 110 μg/mL, 120 μg/mL, 130 μg/mL, 140 μg/mL, 150 μg/mL, 160 μg/mL, 170 μg/mL, 180 μg/mL, 190 μg/mL, 200 μg/mL, 210 μg/mL, 220 μg/mL, 230 μg/mL, 240 μg/mL, 250 μg/mL, 260 μg/mL, 270 μg/mL, 280 μg/mL, 290 μg/mL, and 300 μg/mL. Optionally, the therapeutic protein is present in the mixture at a final concentration greater than about 100 μg/mL or greater than about 200 μg/mL. In some aspects, the therapeutic protein is present in the mixture at a final concentration greater than about 300 μg/mL or even greater than about 500 μg/mL.

The term “serum” as used herein refers to the fraction of blood remaining after clotting proteins and blood cells have been removed. A “depleted fraction of serum” or “depleted fraction” as used herein means a fraction of serum from which one or more components have been removed. The term “non-depleted serum” or “whole serum” is serum from which no components have been removed. In exemplary aspects, the mixture comprises whole serum. In exemplary aspects, the whole serum is human serum, bovine serum (including fetal bovine serum), rabbit serum, mouse serum, rat serum, cynomolgus monkey serum, horse serum, or pig serum. In preferred embodiments, the whole serum is human serum. In exemplary aspects, the depleted fraction of serum is an IgG-depleted serum fraction or a molecular weight range-depleted serum (“depleted fraction serum” or “depleted fraction of serum”). In exemplary aspects, an IgG-depleted serum fraction is one obtained by removing IgG from serum by using Protein A, such as in Protein A affinity chromatography. In exemplary aspects, a depleted fraction of serum is a fraction depleted of molecules having a pre-selected molecular weight range. In exemplary instances, the pre-selected molecular weight range is about 30 kDa to about 300 kDa or higher. In various aspects, the depleted fraction is obtained by size-based filtration or centrifugation or ultra-filtration methods (see, e.g., Kornilov et al., J Extracell Vesicles 7(1): 1422674 (2018). In various aspects, the depleted serum is obtained through commercial vendors, e.g., Thermo Fisher Scientific (Waltham, Mass.), CalBiochem® (Millipore Sigma, Burlington, Mass.), Quidel (San Diego, Calif.), and Complement Technologies (Tyler, Tex.). In exemplary aspects, the depleted fraction is a twice-depleted fraction, optionally, a fraction twice-depleted of IgG or a fraction twice-depleted of molecules having a pre-selected molecular weight. “Twice-depleted” refers to a fraction of serum that has undergone the depletion or removal technique two times.

In exemplary instances, the mixture comprises greater than 80% (v/v) serum or depleted serum optionally, greater than about 85% (v/v) at the beginning of the incubating step (step (a)). In exemplary aspects, the mixture comprises greater than about 90% (v/v) serum or depleted serum at the beginning of the incubating step (step (a)), optionally, about 92% (v/v) to about 98% (v/v) serum or depleted serum, e.g., about 92% (v/v), about 93% (v/v), about 94% (v/v), about 95% (v/v), about 96% (v/v), about 97% (v/v), about 98% (v/v), or even about 99% (v/v), or more. In various aspects, the mixture comprises greater than about 87% (v/v) serum or depleted serum at the beginning of step (a), optionally, greater than about 90% (v/v) serum or depleted serum, such as about 92% to about 98% (v) serum or depleted serum

In exemplary aspects, the sample comprises therapeutic proteins comprising a fluorescent label. In exemplary instances, the method further comprises labeling the therapeutic proteins with a fluorescent label prior to the incubating step (step (a)). The fluorescent label can be in principle any fluorescent label that can be attached, usually via conjugation, to a protein, and in exemplary aspects is selected from the group consisting of fluorescein, rhodamine, hydroxycoumarin, aminocoumarin, methoxycoumarin, Cascade Blue, Pacific Blue, Pacific Orange, Lucifer Yellow, NBD, phycoerythrin (PE), PE-Cy5, PE-Cy7, Red 613 PerCP, TruRed, FluorX, BODIPY-FL, G-Dye100, G-Dye200, G-Dye300, G-Dye400, Cyt, Cy3, Cy3B, Cy3.5, Cy5, Cy5.5, Cy7, TRITC, Lissamine Rhodamine B, Texas Red, allophycocyanin (APC), APC-Cy7, green fluorescent protein (GFP), yellow fluorescent protein (YFP), red fluorescent protein (RFP), and the like. In some aspects, the fluorescent label is any one of the Alexa fluor dyes, e.g., Alexa Fluor 350, Alexa Fluor 405, Alexa Fluor 430, Alexa Fluor 488, Alexa Fluor 500, Alexa Fluor 514, Alexa Fluor 532, Alexa Fluor 546, Alexa Fluor 555, Alexa Fluor 568, Alexa Fluor 594, Alexa Fluor 610, Alexa Fluor 633, Alexa Fluor 635, Alexa Fluor 647, Alexa Fluor 660, Alexa Fluor 680, Alexa Fluor 700, Alexa Fluor 750, or Alexa Fluor 790.

By “incubating” is meant maintaining under conditions favorable for development or reaction. In exemplary aspects, the incubating step (step (a)) comprises incubating the mixture for at least about 1 hour, at least about 2 hours, at least about 3 hours, or at least about 4 hours, optionally, incubating the mixture for at least about 6 hours, at least about 12 hours, at least about 18 hours, or at least about 24 hours. In exemplary aspects, step (a) comprises incubating the mixture for at least about 30 hours, at least about 36 hours, at least about 42 hours, and/or at least about 48 hours, optionally, incubating the mixture for at least about 3 days, at least about 4 days, at least about 5 days, or at least about one week. In exemplary aspects, the incubating step (step (a)) occurs at about 25° C. to 40° C., about 30° C. to about 40° C., or about 35° C. to about 40° C. Optionally, the incubating occurs at about 37° C.±2° C. Additional conditions for the incubating step (step (a)) are described herein as exemplified in the Examples.

In exemplary aspects, the method further comprises a dilution step after the incubating step (step (a)) and before the assaying step (step (b)). Optionally, the mixture is diluted with water or buffer prior to the assaying step (step (b)), and in some aspects, the water or buffer. The buffer can be any one known in the art, including, but not limited to, those listed in Table A.

TABLE A

| Buffer | pKₐ |
|---|---|
| Acetate | 4.8 |
| Succinate | pKₐ₁ = 4.8, pKₐ₂ = 5.5 |
| Citrate | pKₐ₁ = 3.1, pKₐ₂ = 4.8, pKₐ₃ = 6.4 |
| Histidine | 6.0 |
| (imidazole) | |
| Phosphate | pKₐ₁ = 2.15, pKₐ₂ = 7.2, pKₐ₃ = 12.3 |
| TRIS | 8.1 |
| Glycine | pK = 2.35 |

In exemplary aspects, the assaying step (step (b)) comprises assaying the level of HMW species in the mixture, which comprises serum or a depleted fraction thereof, by SEC. In exemplary aspects, the SEC is SEC-high performance liquid chromatography (SEC-HPLC) or SEC Fluorescence (SEC-Fluor) or SEC-UV. Additionally or alternatively, the assaying step (step (b)) comprises other techniques to assay the level of HMW species in the mixture or low molecular weight (LMW; those species that are smaller than the therapeutic protein) species or monomers of the therapeutic protein to ultimately achieve the determination of the level of HMW species in the mixture. The assaying step (step (b)) can include one or more of: mass spectrometry (MS), SEC could be coupled to ultra-high performance liquid chromatography (UHPLC).

In exemplary aspects, the affinity purification is size exclusion chromatography (SEC), affinity chromatography, precipitation using binding target-labeled beads (including precipitation with FcRn-labeled beads), precipitation with cells with on-surface expressed targets (including precipitation with cells with surface expressed FcRn receptors), free flow fractionation (FFF), ion exchange chromatography (IEX), hydrophobic interaction chromatography (HIC), or ultracentrifugation (UC).

In exemplary aspects, the method further comprises a separation step after the incubation step (step (a)) and before the assaying step (step (b)), wherein components of the mixture are separated. In exemplary aspects, components of the mixture are separated by chromatography, optionally, affinity chromatography. Affinity chromatography techniques are known in the art. See, e.g., Handbook of Affinity Chromatography, eds. Hage and Cazes, Taylor and Francis (2005). In alternative aspects, the components of the mixture are separated by another type of chromatography, e.g., anion exchange chromatography, cation exchange chromatography, gel-permeation chromatography, paper chromatography, thin-layer chromatography, gas chromatography, and the like. See, e.g., Coskun, North Clin Istanb 3(2): 156-160 (2016). In exemplary aspects, the affinity chromatography is affinity chromatography with Protein A, Protein L, or an antibody specific for the therapeutic protein or other suitable capture protein. The selection of capture protein used in the affinity chromatography step in some aspects depends on the therapeutic protein. In general aspects, the capture protein binds to the therapeutic protein. In exemplary aspects, when the therapeutic protein is an antibody, antigen-binding fragment of an antibody or an antibody protein product, the capture protein is the antigen to which the therapeutic protein binds. In exemplary aspects, the Protein A or an antibody specific for the therapeutic protein or other suitable capture protein is coupled to the resin to be used in the affinity chromatography column. After the incubation step (step (a)), the mixture is loaded onto the affinity chromatography column or mixed with the resin linked to the capture protein, Protein A, Protein L, or antibody specific for the therapeutic protein, and the fraction comprising the therapeutic protein and HMW species thereof are bound to the resin. In various instances, a resin linked to Protein A, Protein L, or an antibody specific for the therapeutic protein is incubated with the mixture for less than 1 hour, optionally, less than about 30 minutes, less than about 20 minutes, less than about 15 minutes, or less. In various instances, a resin linked to Protein A, Protein L, or an antibody specific for the therapeutic protein is incubated with the mixture for about 5 minutes to about 10 minutes. The bound fraction is eluted off the resin using conditions which can be known or experimentally determined. In exemplary embodiments, the affinity chromatography comprises an elution step comprising eluting with an acidic elution buffer. Optionally, the acidic elution buffer comprises glycine or acetic acid or citrate. In various aspects, the acidic elution buffer has a pH of about 2.5 to about 4.5, optionally, about 2.75 to about 4.0. In exemplary instances, the elution step yields an eluate comprising the therapeutic protein and the method comprises assaying the level of HMW species of the therapeutic protein present in the eluate.

In exemplary aspects, the method further comprises comparing the level(s) of HMW species present in the mixture as assayed in the assaying step (step (b)) to the level of HMW species present in the sample prior to the incubating step (step (a)). Optionally, the level of one or more of dimers, trimers, tetramers, pentamers, hexamers, heptamers, and octamers, of the therapeutic protein present in the mixture as assayed in the assaying step (step (b)) is compared to the level of dimers, trimers, tetramers, pentamers, hexamers, heptamers, or octamers, of the therapeutic protein in the sample prior to the incubating step (step (a)). In exemplary aspects, the method further comprises calculating the percentage of in vivo reversibility of HMW species of the therapeutic protein according to Equation 1:

$$\% in vivo reversibility = {[ 1 - X ]}^{*} 100 \% , wherein \qquad [ Equation 1 ]$$

$$X = \frac{\begin{matrix}\% HMW species of the \\ therapeutic protein present in the mixture\end{matrix}}{\% HMW species in the sample prior to step ( a )} .$$

Accordingly, the present disclosure provides methods of determining the in vivo reversibility of HMW species of a therapeutic protein. The present disclosure provides a method of determining the in vivo reversibility of HMW species of a therapeutic protein, comprising (A) assaying the in vivo level of high molecular weight (HMW) species of a therapeutic protein according to any of the previously described methods, wherein (i) the method further comprises assaying the level of HMW species present in the sample prior to the incubating step (step (a)) or (ii) the level of HMW species present in the sample prior to the incubating step (step (a)) is known and (B) comparing the level(s) of HMW species present in the mixture to the level of HMW species present in the sample prior to the incubating step (step (a)). The present disclosure also provides a method of determining the in vivo reversibility of HMW species of a therapeutic protein, comprising: incubating a mixture comprising a sample comprising the therapeutic protein and a depleted serum, wherein the depleted fraction of serum is a fraction depleted of molecules having a pre-selected molecular weight range, optionally, wherein the pre-selected molecular weight range is about 30 kDa to about 300 kDa or higher, optionally, wherein the depleted fraction is obtained through size-based filtration; assaying the level of HMW species of the therapeutic protein present in the mixture at one or more time points after step (a) by SEC; comparing the level(s) of the HMW species present in the mixture as assayed in step (b) to the level of the HMW species present in the sample prior to step (a); and calculating the percentage of in vivo reversibility of the HMW species of the therapeutic protein. In exemplary aspects, the therapeutic protein has a molecular weight of about 15 kDa or higher. Also, a method of determining the in vivo reversibility of HMW species of a therapeutic protein is provided, wherein the method comprises: incubating a mixture comprising a sample comprising the therapeutic protein and a depleted serum, wherein the depleted serum is an IgG-depleted serum fraction, optionally, obtained by removing IgG from serum by Protein A affinity chromatography; separating components of the mixture by affinity chromatography with a capture molecule to obtain a fraction comprising the therapeutic protein and HMW species thereof; assaying the level of HMW species of the therapeutic protein present in the fraction by SEC, comparing the level(s) of the HMW species present in the fraction as assayed in step (c) to the level of the HMW species present in the sample prior to step (a); and calculating the percentage of in vivo reversibility of the HMW species of the therapeutic protein. In exemplary aspects, the capture molecule is Protein A and the therapeutic protein binds to Protein A, optionally, wherein the therapeutic protein is an antibody, an Fc fusion protein, or an antibody protein product comprising a Protein A binding site. In exemplary aspects, step (b) comprises (i) loading the mixture onto an affinity chromatography column to obtain a bound fraction comprising the therapeutic protein and (ii) eluting the bound fraction off the column. The present disclosure further provides a method of determining the in vivo reversibility of HMW species of a therapeutic protein, comprising: incubating a mixture comprising a sample comprising the therapeutic protein with whole serum, wherein the therapeutic protein comprises a fluorescent label; diluting the mixture; assaying the level of HMW species of the therapeutic protein present in the mixture at one or more time points after step (a) by SEC, comparing the level(s) of the HMW species present in the mixture as assayed in step (c) to the level of the HMW species present in the sample prior to step (a); and calculating the percentage of in vivo reversibility of the HMW species of the therapeutic protein. The present disclosure additionally provides a method of determining the in vivo reversibility of HMW species of a therapeutic protein, comprising: incubating a mixture comprising a sample comprising the therapeutic protein and whole serum; separating components of the mixture by affinity chromatography with a capture molecule to obtain a fraction comprising the therapeutic protein and HMW species thereof; assaying the level of HMW species of the therapeutic protein present in the fraction by SEC, comparing the level(s) of the HMW species present in the fraction as assayed in step (c) to the level of the HMW species present in the sample prior to step (a); and calculating the percentage of in vivo reversibility of the HMW species of the therapeutic protein. In exemplary aspects, the capture molecule is an antibody or a molecule other than an antibody, which binds to the therapeutic protein. In exemplary aspects, the assaying step (step (b)) comprises (i) loading the mixture onto an affinity chromatography column to obtain a bound fraction comprising the therapeutic protein and (ii) eluting the bound fraction off the column. In exemplary aspects, the percentage of in vivo reversibility of the HMW species of the therapeutic protein is calculated according to Equation 1.

Exemplary Therapeutic Proteins

In exemplary aspects, the therapeutic protein is an antibody. As used herein, the term “antibody” refers to a protein having a conventional immunoglobulin format, comprising heavy and light chains, and comprising variable and constant regions. For example, an antibody can be an IgG which is a “Y-shaped” structure of two identical pairs of polypeptide chains, each pair having one “light” (typically having a molecular weight of about 25 kDa) and one “heavy” chain (typically having a molecular weight of about 50-70 kDa). An antibody has a variable region and a constant region. In IgG formats, the variable region is generally about 100-110 or more amino acids, comprises three complementarity determining regions (CDRs), is primarily responsible for antigen recognition, and substantially varies among other antibodies that bind to different antigens. The constant region allows the antibody to recruit cells and molecules of the immune system. The variable region is made of the N-terminal regions of each light chain and heavy chain, while the constant region is made of the C-terminal portions of each of the heavy and light chains. (Janeway et al., “Structure of the Antibody Molecule and the Immunoglobulin Genes”, Immunobiology: The Immune System in Health and Disease, 4^{th} ed. Elsevier Science Ltd./Garland Publishing, (1999)).

The general structure and properties of CDRs of antibodies have been described in the art. Briefly, in an antibody scaffold, the CDRs are embedded within a framework in the heavy and light chain variable region where they constitute the regions largely responsible for antigen binding and recognition. A variable region typically comprises at least three heavy or light chain CDRs (Kabat et al., 1991, Sequences of Proteins of Immunological Interest, Public Health Service N.I.H., Bethesda, Md.; see also Chothia and Lesk, 1987, J. Mol. Biol. 196:901-917; Chothia et al., 1989, Nature 342: 877-883), within a framework region (designated framework regions 1-4, FR1, FR2, FR3, and FR4, by Kabat et al., 1991; see also Chothia and Lesk, 1987, supra).

Antibodies can comprise any constant region known in the art. Human light chains are classified as kappa and lambda light chains. Heavy chains are classified as mu, delta, gamma, alpha, or epsilon, and define the antibody's isotype as IgM, IgD, IgG, IgA, and IgE, respectively. IgG has several subclasses, including, but not limited to IgG1, IgG2, IgG3, and IgG4. IgM has subclasses, including, but not limited to, IgM1 and IgM2. Embodiments of the present disclosure include all such classes or isotypes of antibodies. The light chain constant region can be, for example, a kappa- or lambda-type light chain constant region, e.g., a human kappa- or lambda-type light chain constant region. The heavy chain constant region can be, for example, an alpha-, delta-, epsilon-, gamma-, or mu-type heavy chain constant regions, e.g., a human alpha-, delta-, epsilon-, gamma-, or mu-type heavy chain constant region. Accordingly, in exemplary embodiments, the antibody is an antibody of isotype IgA, IgD, IgE, IgG, or IgM, including any one of IgG1, IgG2, IgG3 or IgG4.

The antibody can be a monoclonal antibody or a polyclonal antibody. In some embodiments, the antibody comprises a sequence that is substantially similar to a naturally-occurring antibody produced by a mammal, e.g., mouse, rabbit, goat, horse, chicken, hamster, human, and the like. In this regard, the antibody can be considered as a mammalian antibody, e.g., a mouse antibody, rabbit antibody, goat antibody, horse antibody, chicken antibody, hamster antibody, human antibody, and the like. In certain aspects, the antibody is a human antibody. In certain aspects, the antibody is a chimeric antibody or a humanized antibody. The term “chimeric antibody” refers to an antibody containing domains from two or more different antibodies. A chimeric antibody can, for example, contain the constant domains from one species and the variable domains from a second, or more generally, can contain stretches of amino acid sequence from at least two species. A chimeric antibody also can contain domains of two or more different antibodies within the same species. The term “humanized” when used in relation to antibodies refers to antibodies having at least CDR regions from a non-human source which are engineered to have a structure and immunological function more similar to true human antibodies than the original source antibodies. For example, humanizing can involve grafting a CDR from a non-human antibody, such as a mouse antibody, into a human antibody. Humanizing also can involve select amino acid substitutions to make a non-human sequence more similar to a human sequence.

An antibody can be cleaved into fragments by enzymes, such as, e.g., papain and pepsin. Papain cleaves an antibody to produce two Fab fragments and a single Fc fragment. Pepsin cleaves an antibody to produce a F(ab′)₂ fragment and a pFc′ fragment. In exemplary aspects of the present disclosure, the therapeutic protein is an antigen binding fragment or an antibody. As used herein, the term “antigen binding antibody fragment” refers to a portion of an antibody that is capable of binding to the antigen of the antibody and is also known as “antigen-binding fragment” or “antigen-binding portion”. In exemplary instances, the antigen binding antibody fragment is a Fab fragment or a F(ab′)₂ fragment.

In various aspects, the therapeutic protein is an antibody protein product. As used herein, the term “antibody protein product” refers to any one of several antibody alternatives which in various instances is based on the architecture of an antibody but is not found in nature. In some aspects, the antibody protein product has a molecular-weight within the range of at least about 12-150 kDa. In certain aspects, the antibody protein product has a valency (n) range from monomeric (n=1), to dimeric (n=2), to trimeric (n=3), to tetrameric (n=4), if not higher order valency. Antibody protein products in some aspects are those based on the full antibody structure and/or those that mimic antibody fragments which retain full antigen-binding capacity, e.g., scFvs, Fabs and VHH/VH (discussed below). The smallest antigen binding antibody fragment that retains its complete antigen binding site is the Fv fragment, which consists entirely of variable (V) regions. A soluble, flexible amino acid peptide linker is used to connect the V regions to a scFv (single chain fragment variable) fragment for stabilization of the molecule, or the constant (C) domains are added to the V regions to generate a Fab fragment [fragment, antigen-binding]. Both scFv and Fab fragments can be easily produced in host cells, e.g., prokaryotic host cells. Other antibody protein products include disulfide-bond stabilized scFv (ds-scFv), single chain Fab (scFab), as well as di- and multimeric antibody formats like dia-, tria- and tetra-bodies, or minibodies (miniAbs) that comprise different formats consisting of scFvs linked to oligomerization domains. The smallest fragments are VHH/VH of camelid heavy chain Abs as well as single domain Abs (sdAb). The building block that is most frequently used to create novel antibody formats is the single-chain variable (V)-domain antibody fragment (scFv), which comprises V domains from the heavy and light chain (VH and VL domain) linked by a peptide linker of \tilde{ }15 amino acid residues. A peptibody or peptide-Fc fusion is yet another antibody protein product. The structure of a peptibody consists of a biologically active peptide grafted onto an Fc domain. Peptibodies are well-described in the art. See, e.g., Shimamoto et al., mAbs 4(5): 586-591 (2012).

Other antibody protein products include a single chain antibody (SCA); a diabody; a triabody; a tetrabody; bispecific or trispecific antibodies, and the like. Bispecific antibodies can be divided into five major classes: BsIgG, appended IgG, BsAb fragments, bispecific fusion proteins and BsAb conjugates. See, e.g., Spiess et al., Molecular Immunology 67(2) Part A: 97-106 (2015).

In exemplary aspects, the therapeutic protein is a bispecific T cell engager (BiTE®) molecule, which is an artificial bispecific monoclonal antibody. Canonical BiTE® molecules are fusion proteins comprising two scFvs of different antibodies. One binds to CD3 and the other binds to a target antigen. BiTE® molecules are known in the art. See, e.g., Huehls et al., Immuno Cell Biol 93(3): 290-296 (2015); Rossi et al., MAbs 6(2): 381-91 (2014); Ross et al., PLoS One 12(8): e0183390.

In exemplary aspects, the therapeutic protein is a chimeric antigen receptor (CAR). Chimeric antigen receptors are genetically engineered fusion proteins constructed from multiple domains typically of other naturally occurring molecules expressed by immune cells. In several aspects, CARs comprises an extracellular antigen-binding domain or antigen recognition domain, a signaling domain and a co-stimulatory domain. CARs are described in the art. See, e.g., Maus et al., Clin Cancer Res 22(8): 1875-1884 (2016); Dotti et al., Immuno Rev (2014) 257(1): 10.1111/imr.12131; Lee et al., Clin Cancer Res (2012): 18(10): 2780-2790; and June and Sadelain, NEJM 379: 64-73 (2018).

Exemplary therapeutic proteins include but are not limited to, CD proteins, growth factors, growth factor receptor proteins (e.g., HER receptor family proteins), cell adhesion molecules (for example, LFA-I, MoI, pI50, 95, VLA-4, ICAM-I, VCAM, and alpha v/beta 3 integrin), hormone (e.g., insulin), coagulation factors, coagulation-related proteins, colony stimulating factors and receptors thereof, and other receptors and receptor-associated proteins or ligands of these receptors, viral antigens.

Exemplary therapeutic proteins include, e.g., any one of the CD proteins, such as CD1a, CD1b, CD1c, CD1d, CD2, CD3, CD4, CD5, CD6, CD7, CD8, CD9, CD10, CD11A, CD11B, CD11C, CDw12, CD13, CD14, CD15, CD15s, CD16, CDw17, CD18, CD19, CD20, CD21, CD22, CD23, CD24, CD25, CD26, CD27, CD28, CD29, CD30, CD31, CD32, CD33, CD34, CD35, CD36, CD37, CD38, CD39, CD40, CD41, CD42a, CD42b, CD42c, CD42d, CD43, CD44, CD45, CD45RO, CD45RA, CD45RB, CD46, CD47, CD48, CD49a, CD49b, CD49c, CD49d, CD49e, CD49f, CD50, CD51, CD52, CD53, CD54, CD55, CD56, CD57, CD58, CD59, CDw60, CD61, CD62E, CD62L, CD62P, CD63, CD64, CD65, CD66a, CD66b, CD66c, CD66d, CD66e, CD66f, CD68, CD69, CD70, CD71, CD72, CD73, CD74, CD75, CD76, CD79a, CD7913, CD80, CD81, CD82, CD83, CDw84, CD85, CD86, CD87, CD88, CD89, CD90, CD91, CDw92, CD93, CD94, CD95, CD96, CD97, CD98, CD99, CD100, CD101, CD102, CD103, CD104, CD105, CD106, CD107a, CD107b, CDw108, CD109, CD114, CD 115, CD116, CD117, CD118, CD119, CD120a, CD120b, CD121a, CDw121b, CD122, CD123, CD124, CD125, CD126, CD127, CDw128, CD129, CD130, CDw131, CD132, CD134, CD135, CDw136, CDw137, CD138, CD139, CD140a, CD140b, CD141, CD142, CD143, CD144, CD145, CD146, CD147, CD148, CD150, CD151, CD152, CD153, CD154, CD155, CD156, CD157, CD158a, CD158b, CD161, CD162, CD163, CD164, CD165, CD166, and CD182.

Exemplary growth factors, include, for instance, vascular endothelial growth factor (“VEGF”), growth hormone, thyroid stimulating hormone (TSH), follicle stimulating hormone (FSH), luteinizing hormone (LH), growth hormone releasing factor (GHRF), parathyroid hormone (PTH), Mullerian-inhibiting substance (MIS), human macrophage inflammatory protein (MIP-I-alpha), erythropoietin (EPO), nerve growth factor (NGF), such as NGF-beta, platelet-derived growth factor (PDGF), fibroblast growth factors (FGF), including, for instance, aFGF and bFGF, epidermal growth factor (EGF), transforming growth factors (TGF), including, among others, TGF-α and TGF-β, including TGF-βI, TGF-β2, TGF-β3, TGF-β4, or TGF-β5, insulin-like growth factors-I and -II (IGF-I and IGF-II), des(I-3)-IGF-I (brain IGF-I), and osteoinductive factors. The therapeutic protein in some aspects is an insulin or insulin-related protein, e.g., insulin, insulin A-chain, insulin B-chain, proinsulin, and insulin-like growth factor binding proteins. Exemplary growth factor receptors include any receptor of any of the above growth factors. In various aspects, the growth factor receptor is a HER receptor family protein (for example, HER2, HER3, HER4, and the EGF receptor), a VEGF receptor, TSH receptor, FSH receptor, LH receptor, GHRF receptor, PTH receptor, MIS receptor, MIP-1-alpha receptor, EPO receptor, NGF receptor, PDGF receptor, FGF receptor, EGF receptor, (EGFR), TGF receptor, or insulin receptor.

Exemplary coagulation and coagulation-related proteins, include, for instance, factor VIII, tissue factor, von Willebrands factor, protein C, alpha-1-antitrypsin, plasminogen activators, such as urokinase and tissue plasminogen activator (“t-PA”), bombazine, thrombin, and thrombopoietin; (vii) other blood and serum proteins, including but not limited to albumin, IgE, and blood group antigens. Colony stimulating factors and receptors thereof, including the following, among others, M-CSF, GM-CSF, and G-CSF, and receptors thereof, such as CSF-1 receptor (c-fms). Receptors and receptor-associated proteins, including, for example, flk2/flt3 receptor, obesity (OB) receptor, LDL receptor, growth hormone receptors, thrombopoietin receptors (“TPO-R,” “c-mpl”), glucagon receptors, interleukin receptors, interferon receptors, T-cell receptors, stem cell factor receptors, such as c-Kit, and other receptors. Receptor ligands, including, for example, OX40L, the ligand for the OX40 receptor. Neurotrophic factors, including bone-derived neurotrophic factor (BDNF) and neurotrophin-3, -4, -5, or -6 (NT-3, NT-4, NT-5, or NT-6). Relaxin A-chain, relaxin B-chain, and prorelaxin; interferons and interferon receptors, including for example, interferon-α, -β, and -γ, and their receptors. Interleukins and interleukin receptors, including IL-I to IL-33 and IL-I to IL-33 receptors, such as the IL-8 receptor, among others. Viral antigens, including an AIDS envelope viral antigen. Lipoproteins, calcitonin, glucagon, atrial natriuretic factor, lung surfactant, tumor necrosis factor-alpha and -beta, enkephalinase, RANTES (regulated on activation normally T-cell expressed and secreted), mouse gonadotropin-associated peptide, DNAse, inhibin, and activin. Integrin, protein A or D, rheumatoid factors, immunotoxins, bone morphogenetic protein (BMP), superoxide dismutase, surface membrane proteins, decay accelerating factor (DAF), AIDS envelope, transport proteins, homing receptors, addressins, regulatory proteins, immunoadhesins, antibodies. Additional exemplary therapeutic proteins include, e.g., myostatins, TALL proteins, including TALL-I, amyloid proteins, including but not limited to amyloid-beta proteins, thymic stromal lymphopoietins (“TSLP”), RANK ligand (“OPGL”), c-kit, TNF receptors, including TNF Receptor Type 1, TRAIL-R2, angiopoietins, and biologically active fragments or analogs or variants of any of the foregoing.

In exemplary aspects, the therapeutic protein is any one of the pharmaceutical agents known as Activase® (Alteplase); alirocumab, Aranesp® (Darbepoetin-alfa), Epogen® (Epoetin alfa, or erythropoietin); Avonex® (Interferon β-Ia); Bexxar® (Tositumomab); Betaseron® (Interferon-β); bococizumab (anti-PCSK9 monoclonal antibody designated as L1L3, see U.S. Pat. No. 8,080,243); Campath® (Alemtuzumab); Dynepo® (Epoetin delta); Velcade® (bortezomib); MLN0002 (anti-α4β7 mAb); MLN1202 (anti-CCR2 chemokine receptor mAb); Enbrel® (etanercept); Eprex® (Epoetin alfa); Erbitux® (Cetuximab); evolocumab; Genotropin® (Somatropin); Herceptin® (Trastuzumab); Humatrope® (somatropin [rDNA origin] for injection); Humira® (Adalimumab); Infergen® (Interferon Alfacon-1); Natrecor® (nesiritide); Kineret® (Anakinra), Leukine® (Sargamostim); LymphoCide® (Epratuzumab); Benlysta™ (Belimumab); Metalyse® (Tenecteplase); Mircera® (methoxy polyethylene glycol-epoetin beta); Mylotarg® (Gemtuzumab ozogamicin); Raptiva® (efalizumab); Cimzia® (certolizumab pegol); Soliris™ (Eculizumab); Pexelizumab (Anti-05 Complement); MEDI-524 (Numax®); Lucentis® (Ranibizumab); Edrecolomab (Panorex®); Trabio® (lerdelimumab); TheraCim hR3 (Nimotuzumab); Omnitarg (Pertuzumab, 2C4); Osidem® (IDM-I); OvaRex® (B43.13); Nuvion® (visilizumab); Cantuzumab mertansine (huC242-DMI); NeoRecormon® (Epoetin beta); Neumega® (Oprelvekin); Neulasta® (Pegylated filgastrim, pegylated G-CSF, pegylated hu-Met-G-CSF); Neupogen® (Filgrastim); Orthoclone OKT3® (Muromonab-CD3), Procrit® (Epoetin alfa); Remicade® (Infliximab), Reopro® (Abciximab), Actemra® (anti-IL6 Receptor mAb), Avastin® (Bevacizumab), HuMax-CD4 (zanolimumab), Rituxan® (Rituximab); Tarceva® (Erlotinib); Roferon-A®-(Interferon alfa-2a); Simulect® (Basiliximab); Stelara™ (Ustekinumab); Prexige® (lumiracoxib); Synagis® (Palivizumab); 14667-CHO (anti-IL15 antibody, see U.S. Pat. No. 7,153,507), Tysabri® (Natalizumab); Valortim® (MDX-1303, anti-B. anthracis Protective Antigen mAb); ABthrax™; Vectibix® (Panitumumab); Xolair® (Omalizumab), ET1211 (anti-MRSA mAb), IL-I Trap (the Fc portion of human IgGI and the extracellular domains of both IL-I receptor components (the Type I receptor and receptor accessory protein)), VEGF Trap (Ig domains of VEGFRI fused to IgGI Fc), Zenapax® (Daclizumab); Zenapax® (Daclizumab), Zevalin® (Ibritumomab tiuxetan), Zetia (ezetimibe), Atacicept (TACI-Ig), anti-α4β7 mAb (vedolizumab); galiximab (anti-CD80 monoclonal antibody), anti-CD23 mAb (lumiliximab); BR2-Fc (huBR3/huFc fusion protein, soluble BAFF antagonist); Simponi™ (Golimumab); Mapatumumab (human anti-TRAIL Receptor-1 mAb); Ocrelizumab (anti-CD20 human mAb); HuMax-EGFR (zalutumumab); M200 (Volociximab, anti-α5β1 integrin mAb); MDX-010 (Ipilimumab, anti-CTLA-4 mAb and VEGFR-I (IMC-18F1); anti-BR3 mAb; anti-C. difficile Toxin A and Toxin B C mAbs MDX-066 (CDA-I) and MDX-1388); anti-CD22 dsFv-PE38 conjugates (CAT-3888 and CAT-8015); anti-CD25 mAb (HuMax-TAC); anti-TSLP antibodies; anti-TSLP receptor antibody (U.S. Pat. No. 8,101,182); anti-TSLP antibody designated as A5 (U.S. Pat. No. 7,982,016); (anti-CD3 mAb (NI-0401); Adecatumumab (MT201, anti-EpCAM-CD326 mAb); MDX-060, SGN-30, SGN-35 (anti-CD30 mAbs); MDX-1333 (anti-IFNAR); HuMax CD38 (anti-CD38 mAb); anti-CD40L mAb; anti-Cripto mAb; anti-CTGF Idiopathic Pulmonary Fibrosis Phase I Fibrogen (FG-3019); anti-CTLA4 mAb; anti-eotaxinI mAb (CAT-213); anti-FGF8 mAb; anti-ganglioside GD2 mAb; anti-sclerostin antibodies (see, U.S. Pat. No. 8,715,663 or 7,592,429) anti-sclerostin antibody designated as Ab-5 (U.S. Pat. No. 8,715,663 or 7,592,429); anti-ganglioside GM2 mAb; anti-GDF-8 human mAb (MYO-029); anti-GM-CSF Receptor mAb (CAM-3001); anti-HepC mAb (HuMax HepC); MEDI-545, MDX-1103 (anti-IFNα mAb); anti-IGFIR mAb; anti-IGF-IR mAb (HuMax-Inflam); anti-IL12/IL23p40 mAb (Briakinumab); anti-IL-23p19 mAb (LY2525623); anti-IL13 mAb (CAT-354); anti-IL-17 mAb (AIN457); anti-IL2Ra mAb (HuMax-TAC); anti-IL5 Receptor mAb; anti-integrin receptors mAb (MDX-OI8, CNTO 95); anti-IPIO Ulcerative Colitis mAb (MDX-1100); anti-LLY antibody; BMS-66513; anti-Mannose Receptor/hCGβ mAb (MDX-1307); anti-mesothelin dsFv-PE38 conjugate (CAT-5001); anti-PDImAb (MDX-1 106 (ONO-4538)); anti-PDGFRα antibody (IMC-3G3); anti-TGFβ mAb (GC-1008); anti-TRAIL Receptor-2 human mAb (HGS-ETR2); anti-TWEAK mAb; anti-VEGFR/Flt-1 mAb; anti-ZP3 mAb (HuMax-ZP3); NVS Antibody #1; NVS Antibody #2; or an amyloid-beta monoclonal antibody.

Additional examples of therapeutic proteins include antibodies shown in Table B and any of infliximab, bevacizumab, cetuximab, ranibizumab, palivizumab, abagovomab, abciximab, actoxumab, adalimumab, afelimomab, afutuzumab, alacizumab, alacizumab pegol, ald518, alemtuzumab, alirocumab, altumomab, amatuximab, anatumomab mafenatox, anrukinzumab, apolizumab, arcitumomab, aselizumab, altinumab, atlizumab, atorolimiumab, tocilizumab, bapineuzumab, basiliximab, bavituximab, bectumomab, belimumab, benralizumab, bertilimumab, besilesomab, bevacizumab, bezlotoxumab, biciromab, bivatuzumab, bivatuzumab mertansine, blinatumomab, blosozumab, brentuximab vedotin, briakinumab, brodalumab, canakinumab, cantuzumab mertansine, cantuzumab mertansine, caplacizumab, capromab pendetide, carlumab, catumaxomab, cc49, cedelizumab, certolizumab pegol, cetuximab, citatuzumab bogatox, cixutumumab, clazakizumab, clenoliximab, clivatuzumab tetraxetan, conatumumab, crenezumab, cr6261, dacetuzumab, daclizumab, dalotuzumab, daratumumab, demcizumab, denosumab, detumomab, dorlimomab aritox, drozitumab, duligotumab, dupilumab, ecromeximab, eculizumab, edobacomab, edrecolomab, efalizumab, efungumab, elotuzumab, elsilimomab, enavatuzumab, enlimomab pegol, enokizumab, enoticumab, ensituximab, epitumomab cituxetan, epratuzumab, erenumab, erlizumab, ertumaxomab, etaracizumab, etrolizumab, evolocumab, exbivirumab, fanolesomab, faralimomab, farletuzumab, fasinumab, fbta05, felvizumab, fezakinumab, ficlatuzumab, figitumumab, flanvotumab, fontolizumab, foralumab, foravirumab, fresolimumab, fulranumab, futuximab, galiximab, ganitumab, gantenerumab, gavilimomab, gemtuzumab ozogamicin, gevokizumab, girentuximab, glembatumumab vedotin, golimumab, gomiliximab, gs6624, ibalizumab, ibritumomab tiuxetan, icrucumab, igovomab, imciromab, imgatuzumab, inclacumab, indatuximab ravtansine, infliximab, intetumumab, inolimomab, inotuzumab ozogamicin, ipilimumab, iratumumab, itolizumab, ixekizumab, keliximab, labetuzumab, lebrikizumab, lemalesomab, lerdelimumab, lexatumumab, libivirumab, ligelizumab, lintuzumab, lirilumab, lorvotuzumab mertansine, lucatumumab, lumiliximab, mapatumumab, maslimomab, mavrilimumab, matuzumab, mepolizumab, metelimumab, milatuzumab, minretumomab, mitumomab, mogamulizumab, morolimumab, motavizumab, moxetumomab pasudotox, muromonab-cd3, nacolomab tafenatox, namilumab, naptumomab estafenatox, narnatumab, natalizumab, nebacumab, necitumumab, nerelimomab, nesvacumab, nimotuzumab, nivolumab, nofetumomab merpentan, ocaratuzumab, ocrelizumab, odulimomab, ofatumumab, olaratumab, olokizumab, omalizumab, onartuzumab, oportuzumab monatox, oregovomab, orticumab, otelixizumab, oxelumab, ozanezumab, ozoralizumab, pagibaximab, palivizumab, panitumumab, panobacumab, parsatuzumab, pascolizumab, pateclizumab, patritumab, pemtumomab, perakizumab, pertuzumab, pexelizumab, pidilizumab, pintumomab, placulumab, ponezumab, priliximab, pritumumab, PRO 140, quilizumab, racotumomab, radretumab, rafivirumab, ramucirumab, ranibizumab, raxibacumab, regavirumab, reslizumab, rilotumumab, rituximab, robatumumab, roledumab, romosozumab, rontalizumab, rovelizumab, ruplizumab, samalizumab, sarilumab, satumomab pendetide, secukinumab, sevirumab, sibrotuzumab, sifalimumab, siltuximab, simtuzumab, siplizumab, sirukumab, solanezumab, solitomab, sonepcizumab, sontuzumab, stamulumab, sulesomab, suvizumab, tabalumab, tacatuzumab tetraxetan, tadocizumab, talizumab, tanezumab, taplitumomab paptox, tefibazumab, telimomab aritox, tenatumomab, tefibazumab, teneliximab, teplizumab, teprotumumab, tezepelumab, TGN1412, tremelimumab, ticilimumab, tildrakizumab, tigatuzumab, TNX-650, tocilizumab, toralizumab, tositumomab, tralokinumab, trastuzumab, TRBS07, tregalizumab, tucotuzumab celmoleukin, tuvirumab, ublituximab, urelumab, urtoxazumab, ustekinumab, vapaliximab, vatelizumab, vedolizumab, veltuzumab, vepalimomab, vesencumab, visilizumab, volociximab, vorsetuzumab mafodotin, votumumab, zalutumumab, zanolimumab, zatuximab, ziralimumab, zolimomab aritox.

TABLE B
Examples of therapeutic antibodies

| Target | HC* Type | | | LC* SEQ | HC* SEQ |
|---|---|---|---|---|---|
| (informal name) | (including allotypes) | LC* Type | pl | ID NO: | ID NO: |
| anti-amyloid | IgG1 (f) (R;EM) | Kappa | 9.0 | 2 | 3 |
| GMCSF (247) | IgG2 | Kappa | 8.7 | 4 | 5 |
| CGRPR | IgG2 | Lambda | 8.6 | 6 | 7 |
| RANKL | IgG2 | Kappa | 8.6 | 8 | 9 |
| Sclerostin (27H6) | IgG2 | Kappa | 6.6 | 10 | 11 |
| IL-1R1 | IgG2 | Kappa | 7.4 | 12 | 13 |
| Myostatin | IgG1 (z) (K;EM) | Kappa | 8.7 | 14 | 15 |
| B7RP1 | IgG2 | Kappa | 7.7 | 16 | 17 |
| Amyloid | IgG1 (za) (K;DL) | Kappa | 8.7 | 18 | 19 |
| GMCSF (3.112) | IgG2 | Kappa | 8.8 | 20 | 21 |
| CGRP (32H7) | IgG2 | Kappa | 8.7 | 22 | 23 |
| CGRP (3B6.2) | IgG2 | Lambda | 8.6 | 24 | 25 |
| PCSK9 (8A3.1) | IgG2 | Kappa | 6.7 | 26 | 27 |
| PCSK9 (492) | IgG2 | Kappa | 6.9 | 28 | 29 |
| CGRP | IgG2 | Lambda | 8.8 | 30 | 31 |
| Hepcidin | IgG2 | Lambda | 7.3 | 32 | 33 |
| TNFR p55) | IgG2 | Kappa | 8.2 | 34 | 35 |
| OX40L | IgG2 | Kappa | 8.7 | 36 | 37 |
| HGF | IgG2 | Kappa | 8.1 | 38 | 39 |
| GMCSF | IgG2 | Kappa | 8.1 | 40 | 41 |
| Glucagon R | IgG2 | Kappa | 8.4 | 42 | 43 |
| GMCSF (4.381) | IgG2 | Kappa | 8.4 | 44 | 45 |
| Sclerostin (13F3) | IgG2 | Kappa | 7.8 | 46 | 47 |
| CD-22 | IgG1 (f) (R;EM) | Kappa | 8.8 | 48 | 49 |
| INFgR | IgG1 (za) (K;DL) | Kappa | 8.8 | 50 | 51 |
| Ang2 | IgG2 | Kappa | 7.4 | 52 | 53 |
| TRAILR2 | IgG1 (f) (R;EM) | Kappa | 8.7 | 54 | 55 |
| EGFR | IgG2 | Kappa | 6.8 | 56 | 57 |
| IL-4R | IgG2 | Kappa | 8.6 | 58 | 59 |
| IL-15 | IgG1 (f) (R;EM) | Kappa | 8.8 | 60 | 61 |
| IGF1R | IgG1 (za) (K;DL) | Kappa | 8.6 | 62 | 63 |
| IL-17R | IgG2 | Kappa | 8.6 | 64 | 65 |
| Dkk1 (6.37.5) | IgG2 | Kappa | 8.2 | 66 | 67 |
| Sclerostin | IgG2 | Kappa | 7.4 | 68 | 69 |
| TSLP | IgG2 | Lambda | 7.2 | 70 | 71 |
| Dkk1 (11H10) | IgG2 | Kappa | 8.2 | 72 | 73 |
| PCSK9 | IgG2 | Lambda | 8.1 | 74 | 75 |
| GIPR (2G10.006) | IgG1 (z) (K;EM) | Kappa | 8.1 | 76 | 77 |
| Activin | IgG2 | Lambda | 7.0 | 78 | 79 |
| Sclerostin (2B8) | IgG2 | Lambda | 6.7 | 80 | 81 |
| Sclerostin | IgG2 | Kappa | 6.8 | 82 | 83 |
| c-fms | IgG2 | Kappa | 6.6 | 84 | 85 |
| α4β7 | IgG2 | Kappa | 6.5 | 86 | 87 |
| PD-1 | IgG2 | Kappa | — | 87 | 88 |

*HC—antibody heavy chain; LC—antibody light chain.

In some embodiments, the therapeutic polypeptide is a BiTE® molecule. Blinatumomab (BLINCYTO®) is an example of a BiTE® molecule, specific for CD19. BiTE® molecules that are modified, such as those modified to extend their half-lives, can also be used in the disclosed methods.

The following examples are given merely to illustrate the present invention and not in any way to limit its scope.

### EXAMPLES

The following examples describe an exemplary method of assaying the in vivo reversibility of HMW species of a therapeutic protein. In each example, a sample of a therapeutic protein was added to a sample comprising either serum or a depleted fraction of serum to form a mixture and the mixture was incubated at 37° C. with gentle orbital motion (200 rpm) over the course of up to three days. Aliquots of the mixture were taken during the incubation period at 0 hours, 1 hour, 4 or 6 hours, 1 day, 2 days, and 3 days. The aliquots were then used for assaying levels of HMW species by SEC-HPLC. Changes in the target molecule's HMW level and profile were analyzed. The percentage of in vivo reversibility of HMW species of the therapeutic protein was calculated according to Equation 1 described herein.

In these studies, two therapeutic proteins were tested: Therapeutic Protein 1 (TP1) was a mouse/human chimeric antibody and Therapeutic Protein 2 (TP2) was an IgG2 antibody.

Prior to assaying the in vivo reversibility of HMW species of these therapeutic proteins, initial steps were taken to enrich the % HMW species in the therapeutic protein samples (prior to being added to serum or depleted serum) and this was done by SEC-Semi-Preparative HPLC. Through this technique, the % HMW species of TP1 was determined as 4.6% and the % HMW species of TP2 was determined as 53%. Because the % HMW species of TP2 was so high, the therapeutic fraction was diluted with a solution comprising TP2 monomers with less than 0.5% HMW species to a solution comprising 5% HMW species or a solution comprising 10% HMW species. The diluted samples of TP2 (5% HMW species, 10% HMW species) were used in the methods of assaying the in vivo reversibility of HMW species. Because TP1 was determined to have only 4.6% HMW species, the TP1 sample could be used without any dilution step.

### Example 1A

This example demonstrates a first exemplary method of the present disclosure called Large Protein Depleted Human Serum (LPDS) method.

In this example, the sample comprising a therapeutic protein was added to a sample comprising a depleted fraction of serum to form a mixture. The depleted fraction of serum was obtained by pooling normal human serum samples and subjecting the pooled serum to size-based centrifugal filtration to remove large proteins greater than 30 kDa. Briefly, serum was transferred into new 0.5-mL capacity Amicon® concentrator units with 30 kDa molecular weight cutoff. The filter units were centrifuged at \tilde{ }14,000 rcf for 15 minutes to generate large protein-depleted (LPD) filtrates. The LPD filtrates were subjected to a second round of filtration using the same conditions. The twice-depleted filtrates were pooled, aliquoted, stored at 4° C. and used within 4 weeks. The twice-depleted filtrates were analyzed by UV VIS-spectroscopy using a SOLO VPE system (Fuji Film Diosynthe Biotechnologies, Morrisville, N.C.) to determine the components of the twice-depleted fraction of serum. Based on this analysis, the twice-depleted fraction of serum was found to contain both inorganic and organic components, and 4-5% small proteins from human serum (relative to the non-depleted serum).

A sample of TP1 (determined to have an initial % HMW species of 4.6%) was added to a sample of the twice-depleted fraction of serum to form a mixture. The mixture was greater than 97% (v/v) twice-depleted fraction and the final concentration of TP1 in the mixture was 250 μg/mL. The mixture was incubated as described above and aliquots of the mixture were taken at various time points during the incubation period. The aliquots were then used for assaying levels of HMW species by SEC-HPLC. The percentage of in vivo reversibility of HMW species of the therapeutic protein was calculated according to Equation 1 described herein and the results are shown in Table 1.

TABLE 1

| Time | HMW | HMW |
|---|---|---|
| (hr) | % | reversibility % |
| 0 | 4.62 | NA |
| 1 | 4.30 | 7% |
| 4 | 3.96 | 14% |
| 8 | 3.57 | 23% |
| 24 | 3.18 | 31% |
| 42 | 3.13 | 32% |
| 48 | 2.87 | 38% |
| 72 | 2.68 | 42% |

As shown in Table 1, the HMW species of TP1 showed up to 42% reversibility at the 72 hour time point. After this time, % reversibility plateaued.

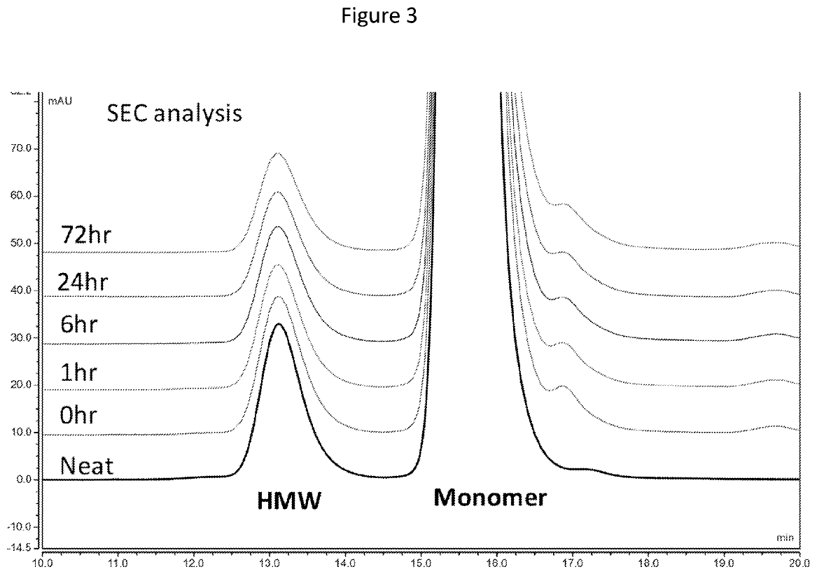

The same protocol was followed for TP2, except that the sample of TP2 was diluted prior to being added to the depleted serum, as described above. Two diluted fractions of TP2 were used in this study: a first having a % HMW species diluted to 5%, and a second having a % HMW species diluted to 10%. Each diluted fraction was added to the twice-depleted fraction to obtain a mixture having greater than 99% (v/v) twice-depleted fraction and wherein the final concentration of TP2 in the mixture was 250 μg/mL. The results for the mixture comprising 5% HMW species are shown in Table 2. The SEC chromatograms are shown in FIG. 3.

TABLE 2

| Time | HMW | HMW |
|---|---|---|
| (hr) | % | reversibility % |
| Initial Neat | 5.20 | NA |
| 0 | 4.47 | 14% |
| 1 | 4.01 | 23% |
| 6 | 3.73 | 28% |
| 24 | 3.24 | 38% |
| 48 | 3.02 | 42% |
| 72 | 2.99 | 43% |

As shown in Table 2, the HMW species of TP2 showed up to 43% reversibility at 72 hour time point. After this time, the % reversibility plateaued.

Additional results for both diluted samples of TP2 are shown in Table 3.

TABLE 3

| Initial % | Diluted % | % reversibility |
|---|---|---|
| HMW* | HMW* | of HMW |
| 53 | 5 | 43 |
| 53 | 10 | 33 |

*% HMW species of TP2 samples prior to being added to depleted serum.

The above example demonstrated a method of determining the % reversibility of the HMW species of two different therapeutic proteins.

### Example 1B

This example demonstrates an exemplary method of determining the % reversibility of the HMW species of a BiTE® molecule protein.

The reversibility of HMW species in a canonical BiTE® molecule with anti-CD3 and tumor target binding domain was evaluated in a serum-like environment. In this example, large protein-depleted serum (LPDS) was used as essentially described in Example 1A. Briefly, a sample of therapeutic protein (TP3) having a canonical BiTE® molecule structure comprising an anti-CD3 and a tumor target binding domain (determined to have an initial % HMW species of 5.76%) was added to a sample of the twice-depleted fraction of serum to form a mixture. The mixture was greater than 87% (v/v) twice-depleted fraction and the final concentration of TP3 in the mixture was 100 micrograms/m L. The mixture was incubated as described in Example 1A and aliquots of the mixture were taken at various time points during the incubation period. The aliquots were then used for assaying levels of HMW species by SEC-HPLC. The results are shown in Table 4.

TABLE 4

| Time | Total | HMW |
|---|---|---|
| (hr) | HMW % | reversibility % |
| Initial Neat | 5.76 | 0 |
| 0.05 | 5.44 | 6 |
| 1 | 5.07 | 12 |
| 6 | 2.77 | 52 |
| 24 | 0.79 | 86 |

As shown in Table 4, the HMW species of TP3 showed up to 86% reversibility at 24 hour time point. After this time, the % reversibility plateaued.

This example demonstrated that the LPDS method can be used to determine the reversibility of a canonical BiTE® molecule.

### Example 2

This example demonstrates a second exemplary method of the present disclosure called IgG Depleted Human Serum (IgGDS) method.

As in Example 1, the sample comprising a therapeutic protein was added to a sample comprising a depleted fraction of serum to form a mixture. However, the depleted fraction was an endogenous immunoglobulin-depleted fraction of serum obtained by subjecting pooled normal human serum to Protein A affinity chromatography. Briefly, Protein A resin (MabSuRE Select LX, GE Healthcare) was transferred into an empty spin column and conditioned with binding buffer, 20 mM Tris, 150 mM NaCl, pH 7. Pooled human serum was added into the column, and the column was subjected to slow, gentle mixing by lab rotator for 10 minutes to promote interaction between Protein A and serum immunoglobulin. Afterward, the column was centrifuged to collect IgG-depleted serum filtrate. The resin was regenerated by 0.1% acetic acid and reconditioned, and the IgG-depleted serum filtrate was subjected to a second round of IgG-depletion following the same steps. The twice-depleted serum filtrate was analyzed by SEC-HPLC to confirm IgG-depletion. Upon confirmation, the twice-depleted fraction of serum was aliquoted and stored frozen at −20° C.

A sample of TP1 (determined to have an initial % HMW species of 4.6%) was added to a sample of the twice-Ig-depleted fraction of serum to form a mixture. The mixture was greater than 97% (v/v) twice-Ig-depleted fraction and the final concentration of TP1 in the mixture was 250 μg/mL. The mixture was incubated as described above and aliquots of the mixture were taken during the incubation period. As shown by protein concentration analysis, the IgG-depleted fraction mostly consisted of serum components other than native IgGs. It was determined that a purification step was needed before SEC analysis.

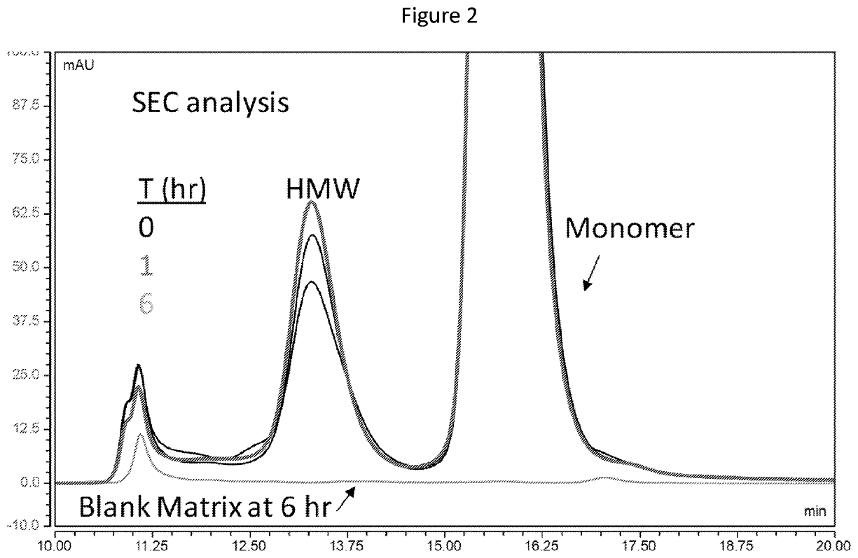

Accordingly, prior to assaying the level of HMW species of the therapeutic protein by SEC, the mixture was subjected to Protein A chromatography to isolate the desired fraction containing the HMW species. Briefly, Protein A resin was transferred into an empty spin column and conditioned with binding buffer. The sample containing the mixture (comprising depleted serum and therapeutic protein) was added to the column, and the column was subjected to slow, gentle mixing by lab rotator for 10 minutes to promote interaction between Protein A and therapeutic protein. Afterward, the column was centrifuged, then washed thoroughly with binding buffer to flush out residual non-binding serum components. Resin-bound species were then eluted by acidic elution using 0.1% acetic acid in several small-volume fractions. Fraction concentrations were measured to determine therapeutic protein and HMW species content, and HMW species-containing fractions were pooled. The pooled HMW species-containing fractions were then used for assaying levels of HMW species by SEC-HPLC. The percentage of in vivo reversibility of HMW species of the therapeutic protein was calculated according to Equation 1 described herein. FIG. 2 provides an overlay of the SEC chromatograms of TP2 diluted to 10% over the incubation time course. As shown in FIG. 2, the % HMW species of TP2 decreased over time. Results for each therapeutic protein (TP1 and TP2) are shown in Table 5.

TABLE 5

| | TP1 HMW % | | |
|---|---|---|---|
| Time | reversibility | TP2 HMW % reversibility | |
| (hr) | 5% HMW | 5% HMW | 10% HMW |
| 0 | 0% | 9% | 14% |
| 1 | 0% | 11% | 25% |
| 4 or 6 | 12% | NA | 26% |

As shown in Table 3, the calculated percentage of in vivo reversibility of HMW species of the TP1 was about 12% (at the 4 hour timepoint). The calculated percentages of in vivo reversibility of HMW species of the TP2 were 11% reversibility (at the 1 hr timepoint) for the TP2 sample diluted to 5% HMW species and 26% reversibility (at the 6 hr timepoint) for the TP2 sample diluted to 10% HMW species.

The above example demonstrated a method of determining the % reversibility of the HMW species for two different therapeutic proteins. Here, the method can be used for any Fc-containing therapeutic protein.

### Example 3

This example demonstrates a third exemplary method of the present disclosure called whole serum with fluorescence labeling (WSFL) method.

In this method, the sample comprising a therapeutic protein was labeled with a fluorescent label. Briefly, enriched HMW species of the therapeutic protein were first labeled with Alexa Fluor™ 488 labeling kit following the manufacture instructions. The labeled fractions were washed using 0.5 mL capacity Amicon® concentrator units with a 30 kDa molecular weight cutoff. The protein concentration was then measured by a spectrophotometer following the manufacture instructions.

A sample comprising the labeled HMW species was added to a sample comprising whole serum (a serum that has not been through any depletion step) to obtain a mixture. The concentration of the therapeutic protein in the mixture was 250 μg/mL and the whole serum in the mixture was greater than 90% (v/v). The mixture was incubated as essentially described above and aliquots taken throughout the time course were obtained for analysis by SEC-HPLC-FLD. The % HMW species was used to calculate the % reversibility of the HMW species of the therapeutic protein.

Following this method, a sample of TP1 demonstrated % reversibility of less than 10% up to 6 hours.

The above example demonstrated a method of determining the % reversibility of the HMW species for two different therapeutic proteins. This method can be used for any type of therapeutic protein.

### Example 4

This example demonstrates a fourth exemplary method of the present disclosure called Whole Serum with Antibody Capture (WSAC) method.

This method is similar to the WSFL method in that whole serum is used. This method is also similar to the IgGDS method in that a separation step is performed prior to SEC.

In this method, a sample comprising the HMW species was added to a sample comprising whole serum (a serum that has not been through any depletion step) to obtain a mixture. The concentration of the therapeutic protein in the mixture was 250 μg/mL and the mixture was greater than 97% (v/v) whole serum. The mixture was then incubated as described above and aliquots of the mixture were taken at various time points during the incubation period. Separation of components of aliquots of the mixture was carried out by affinity chromatography using therapeutic protein-specific antibody that is covalently coupled to sepharose resin. The separation step allowed for the desired fraction containing the HMW species to be isolated. Generation of the antibody-coupled resin was generated as described below. Once the affinity chromatography column was set up, the aliquot of the mixture (comprising serum and therapeutic protein) was added to the column, and the column was subjected to slow, gentle mixing by lab rotator for 10 minutes to promote interaction between the antibody-coupled resin and the therapeutic protein. Afterward, the column was centrifuged, then washed thoroughly with binding buffer to flush out residual non-binding serum components. Resin-bound species were then eluted by acidic elution using 100 mM glycine at pH 3.0 in several small-volume fractions. Fraction concentrations were measured to determine therapeutic protein and HMW species content, and HMW species-containing fractions were pooled. The pooled HMW species-containing fractions were then used for assaying levels of HMW species by SEC-HPLC. The percentage of in vivo reversibility of HMW species of the therapeutic protein was calculated according to Equation 1 described herein.

Generation of the antibody-coupled resin: Briefly, the activated resin was transferred into an empty spin column and conditioned with inert buffer. Coupling reagent and anti-therapeutic protein antibody were added into the column at or close to manufacturer-prescribed concentrations, and the column was subjected to slow, gentle mixing by lab rotator to promote the coupling reaction. Concentration of the free antibody (the antibody specific to the therapeutic protein) was measured at 1+-hour intervals to monitor coupling progress. The reaction was performed at room temperature. If the reaction needed to be extended overnight, the reaction setup was transferred into a 5° C.-cold room. Once coupling was completed (as indicated by a plateau in free anti-therapeutic protein antibody concentration), the column was centrifuged to remove the reaction solution, then washed thoroughly with inert buffer. Resin was then subjected to a second coupling reaction with ethanolamine and coupling reagent to block any remaining active coupling sites in resin. The column was centrifuged and washed thoroughly as described previously and stored in inert buffer with sodium azide.

In this method, the reversibility of HMW species was assessed in whole serum directly. The samples were isolated from serum through immune-based capture using an antibody specific for the therapeutic protein coupled to resin. Following separation using the antibody coupled resin, SEC analysis was performed to measure the amount of HMW species. The percentage of in vivo reversibility of HMW species of the therapeutic protein was calculated according to Equation 1 described herein. Results for TP1 are shown in Table 6.

TABLE 6

| Time | HMW |
|---|---|
| (hr) | reversibility % |
| 0 | 0% |
| 1 | 3% |
| 6 | 9% |

The HMW species of TP1 (which were initially 4.6% prior to being added to serum) showed a 9% reversibility up to 6 hours.

The above example demonstrated a method of determining the % reversibility of the HMW species for a therapeutic protein. This method can be used for any type of therapeutic protein.

### Example 5

This example demonstrates an alternative way of performing the method described in Example 2.

A therapeutic protein having a canonical BiTE® molecule structure with single chain variable domains, but without Fc was used in a serum reversibility study using the depleted serum. The method was similar to that described in Example 2 except that Protein L was used instead of Protein A. Unlike Protein A, Protein L binds antibodies through kappa light chain interactions. Protein L binds to all antibody classes (including IgG, IgM, IgA, IgE, and IgD), single chain variable fragments (scFvs), and Fab fragments. After Protein L depletion, all antibodies and antibody fragments with Kappa light chains are eliminated in the final serum matrix for the reversibility study.

In the first step of this method, a depleted serum fraction was prepared by removing all components that bind to Protein L from serum. This depleted serum fraction was prepared using a Protein L resin. A sample of TP3 (described in Example 1B; determined to have an initial % HMW species of 5.28%) was added to the prepared depleted serum fraction to form a mixture. The mixture was greater than 87% (v/v) depleted serum fraction and the final concentration of TP3 in the mixture was 100 micrograms/mL. TP3 was then incubated in this depleted serum fraction for different time points. The mixture was subjected to Protein L chromatography to isolate the desired fraction containing the HMW species of TP3. For this particular therapeutic protein, two acidic elution buffers have been tested: 0.1% acetic acid and 50 mM sodium acetate (pH 3.3). The latter maintains HMW % for the therapeutic protein with better recovery from the initial evaluations. Finally, the level of HMW species of the TP3 was assayed by SEC. The results are shown in Table 7.

TABLE 7

| Time | | HMW |
|---|---|---|
| (hr) | HMW | reversibility % |
| Initial Neat | 5.28 | 0 |
| 0.05 | 5.20 | 2 |
| 1 | 4.48 | 15 |
| 6 | 1.48 | 72 |
| 24 | 0.64 | 88 |

This example demonstrates that the methods of the present disclosure may be used for testing in vivo reversibility of many types of therapeutic proteins. This example also demonstrates that the protein L method can be used to evaluate the reversibility of BiTE® molecules, and it has generally the same experimental design as IgG depleted method.

### Example 6

This example demonstrates the WSAC method with varied mixing times during the immunoseparation step described in Example 4.

The WSAC method described in Example 4 was carried out except that mixing times were varied during the separation of a therapeutic protein by affinity chromatography using therapeutic protein-specific antibody that is covalently coupled to sepharose resin. In this experiment, a therapeutic protein sample was mixed with whole serum and an aliquot of the mixture (comprising serum and therapeutic protein) was added to a spin column comprising resin attached to an antibody specific for the therapeutic protein. The column with the aliquot was subjected to slow, gentle mixing by lab rotator for about 5 min, 30 min, or about 2 hours. After centrifugation to separate non-binding components of the aliquot from the resin, the spin columns were washed with a wash buffer comprising DPBS or 0.5 M NaCl. Before allowing the wash buffer to elute from the 5-min spin column, the spin column was subjected to multiple gentle inversions.

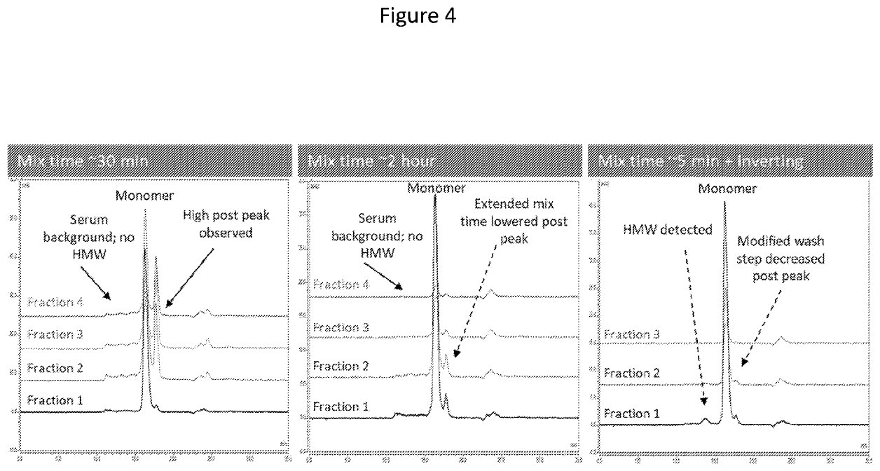

As shown in FIG. 4, a 5-min mixing time with gentle physical inversion of the spin column during the wash step was sufficient to detect HMW species and capture the therapeutic protein with minimal post peak thought to be a co-eluting serum component. In contrast, the 30-min and 2-hour mixing times without gentle physical inversion during the wash step led to high post-peak, no detection of HMW species, and longer sample process compared with the 5-min mixing time.

These results support that a 5-minute mixing time between resin and mixture is sufficient for purposes of binding the therapeutic proteins/HMW species thereof to the resin.

### Example 7

This example demonstrates a series of experiments conducted to identify suitable elution conditions during the immunoseparation step described in both example 2 and example 4.

The ratio of affinity resin and elution volume was explored with the goal that the eluate could be loaded directly for SEC-HPLC analysis without a concentration step, which could induce HMW species formation. Protein A resin solution (100 μL or 200 μL) was transferred into 2-mL disposable spin columns. Test samples were added to a column and binding was allowed to take place by gentle mixing column for 10 min with lab rotator. The samples tested included 1 mL IgG-depleted serum with or without therapeutic protein (250 μg) or 1 mL water with therapeutic protein (250 μg). The resin was subsequently washed with inert buffer to remove non-binding components. The resin-bound components were released using eluting buffer in 100 μL fractions. Each fraction was subjected to UV-VIS to determine the protein concentration for each fraction. The protein concentrations for each fraction are shown in Table 8.

TABLE 8

| | Protein Concentration (mg/mL) | | |
|---|---|---|---|
| | 200 μl Protein A resin | 200 μl Protein A resin | 100 μl Protein A resin |
| | IgG depleted serum | IgG depleted serum | Water with |
| Fraction | with therapeutic protein | without therapeutic protein | therapeutic protein |
| 1 | 0.100 | 0.095 | 0.184 |
| 2 | 0.093 | 0.076 | 1.522 |
| 3 | 1.242 | 0.043 | 0.502 |
| 4 | NA | NA | 0.056 |
| 5 | NA | NA | 0.020 |

The results of this experiment support that more eluting buffer was needed to completely release the therapeutic protein when 200 μL Protein A resin was used. Release was evident at Fraction 3. When 100 μL Protein A resin was used, the first three fractions eluted the majority of bound target, that, when pooled, provided sufficiently high concentration of protein for SEC-HPLC analysis.

The eluting buffers used in the method of Example 4 must be capable of releasing the binding between therapeutic protein and capture antibody without inducing HMW formation or degradation. In a related study, elution buffers used in the WSAC method were evaluated for both TP1 and TP2. For TP1, components of elution buffer and the pH thereof were tested by using 0.1% acetic acid, glycine (pH 3.0), glycine (pH 2.3), or glycine (pH 2.0) to release resin-bound components in 1-mL or 0.5 mL fractions. Briefly, capture antibody resin (200 μL) specific to TP1 was added to a 2-mL disposable spin column and conditioned with inert buffer. Test samples each comprising 250 μg TP1 in 1-mL DPBS were added to spin columns. The columns were then gently mixed using a lab rotator. Resin-bound components were released with elution buffer and fractions were collected. SEC-HPLC was carried out on the fractions.

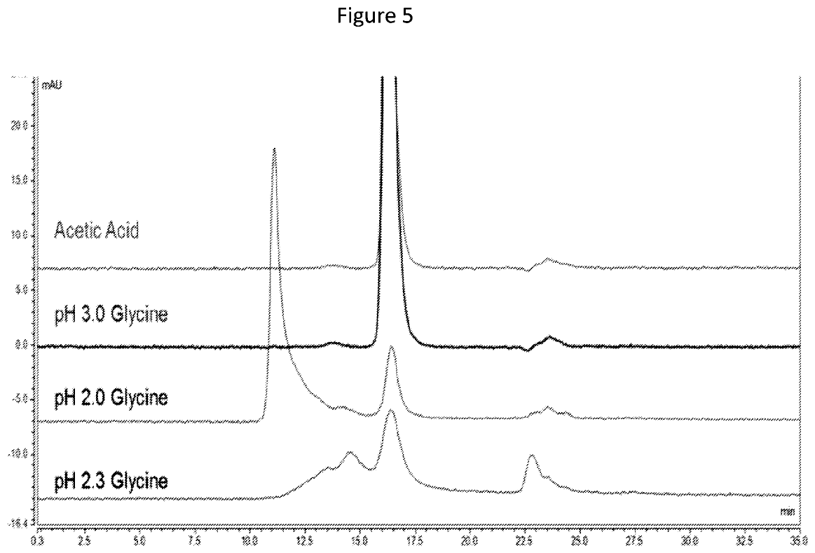

The results are shown in FIG. 5. The two glycine buffers with lower pH induced formation of higher-order HMW (HHMW) species. Glycine at pH 3.0 and acetic acid buffers displayed HMW profiles similar to controls. These data support the use of the glycine pH 3.0 and acetic acid buffers as elution buffers for the first therapeutic protein.

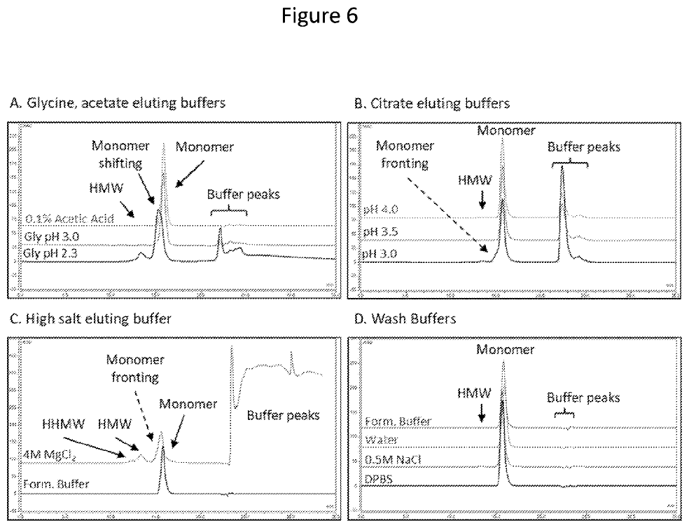

For TP2, a sample of TP2 (250 μg) was spiked into one of many elution buffers tested and kept at room temperature for more than two hours. The samples were then evaluated by SEC-HPLC using the platform SEC-HPLC method. The elution buffers tested were glycine (pH 2.3), glycine (pH 3.0), 0.1% acetic acid, citrate (pH 3.0), citrate (pH 3.5), citrate (pH 4.0), and 4 M MgCl₂. SEC-HPLC analysis was performed on the different spiked elution buffers. Spectra are shown in FIG. 6. Wash buffers and formulation buffer were also spiked with TP2 and analyzed by SEC-HPLC in the same manner as the elution buffers. Tested wash buffers included DPBS, 0.5 M NaCl, formulation buffer and water. The SEC-HPLC spectra using the different wash buffers are shown in FIG. 6.

Of the tested eluting buffers, acetic acid, citrate (pH 3.5), and citrate (pH 4.0) worked well in preventing denaturation of the therapeutic protein. The other eluting buffers tested induced aggregation (increased high molecular weight species or HMW, generation of higher order high molecular weight species or HHMW, and fronting of the monomer which indicates potential presence of unresolved HMW), indicating that these buffers denature TP2. Finally, all wash buffers tested did not denature TP2.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the disclosure (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted.

Recitation of ranges of values herein are merely intended to serve as a shorthand method of referring individually to each separate value falling within the range and each endpoint, unless otherwise indicated herein, and each separate value and endpoint is incorporated into the specification as if it were individually recited herein.

All methods described herein can be performed in any suitable order unless otherwise indicated herein or otherwise clearly contradicted by context. The use of any and all examples, or exemplary language (e.g., “such as”) provided herein, is intended merely to better illuminate the disclosure and does not pose a limitation on the scope of the disclosure unless otherwise claimed. No language in the specification should be construed as indicating any non-claimed element as essential to the practice of the disclosure.

Preferred embodiments of this disclosure are described herein, including the best mode known to the inventors for carrying out the disclosure. Variations of those preferred embodiments can become apparent to those of ordinary skill in the art upon reading the foregoing description. The inventors expect skilled artisans to employ such variations as appropriate, and the inventors intend for the disclosure to be practiced otherwise than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

What is claimed:

1. An in vitro method of assaying an in vivo level of high molecular weight (HMW) species of a therapeutic protein, said method comprising:
a. incubating a mixture comprising (i) a sample comprising the therapeutic protein and (ii) serum, or a depleted fraction thereof; and
b. assaying the level of HMW species of the therapeutic protein present in the mixture at one or more time points after step (a),
optionally, wherein (i) the size of the HMW species assayed are less than about 0.1 microns in size, (ii) the level of HMW species of the therapeutic protein in the mixture is assayed by size-exclusion chromatography (SEC), or (iii) both (i) and (ii).

2. The in vitro method of claim 1, wherein the size of the HMW species assayed are less than 99 nm in size, optionally, about 10 nm to about 99 nm in size.

3. The in vitro method of claim 1 or 2, wherein the HMW species comprise one or more of dimers, trimers, tetramers, pentamers, hexamers, heptamers, and octamers, of the therapeutic protein.

4. The in vitro method of any one of claims 1 to 3, wherein (i) the method further comprises assaying the level of HMW species present in the sample prior to step (a) or (ii) the level of HMW species present in the sample prior to step (a) is known.

5. The in vitro method of any one of claims 1 to 4, wherein (i) the method further comprises assaying the level of one or more of dimers, trimers, tetramers, pentamers, hexamers, heptamers, and octamers, of the therapeutic protein prior to step (a), or (ii) the level of one or more of dimers, trimers, tetramers, pentamers, hexamers, heptamers, and octamers, of the therapeutic protein present in the sample prior to step (a) is known.

6. The in vitro method of any one of claims 1 to 5, wherein step (b) comprises assaying the level of each of dimers, trimers, tetramers, pentamers, hexamers, heptamers, or octamers, of the therapeutic protein.

7. The in vitro method of any one of the preceding claims, further comprising comparing the level(s) of HMW species present in the mixture as assayed in step (b) to the level of HMW species present in the sample prior to step (a), optionally, wherein the level of one or more of dimers, trimers, tetramers, pentamers, hexamers, heptamers, and octamers, of the therapeutic protein present in the mixture as assayed in step (b) is compared to the level of dimers, trimers, tetramers, pentamers, hexamers, heptamers, or octamers, of the therapeutic protein in the sample prior to step (a).

8. The in vitro method of any one of the preceding claims, further comprising calculating the percentage of in vivo reversibility of HMW species of the therapeutic protein according to Equation 1:
$$\% in vivo reversibility = {[ 1 - X ]}^{*} 100 \% , wherein \qquad [ Equation 1 ]$$
$$X = \frac{\begin{matrix}\% HMW species of the \\ therapeutic protein present in the mixture\end{matrix}}{\% HMW species in the sample prior to step ( a )} .$$

9. The in vitro method of any one of the preceding claims, wherein step (a) comprises incubating the mixture for at least about 1 hour, at least about 2 hours, at least about 3 hours, or at least about 4 hours, optionally, incubating the mixture for at least about 6 hours, at least about 12 hours, at least about 18 hours, or at least about 24 hours.

10. The in vitro method of any one of the preceding claims, wherein step (a) comprises incubating the mixture for at least about 30 hours, at least about 36 hours, at least about 42 hours, and/or at least about 48 hours, optionally, incubating the mixture for at least about 3 days, at least about 4 days, at least about 5 days, or at least about one week.

11. The in vitro method of any one of the preceding claims, wherein the therapeutic protein is a recombinant protein.

12. The in vitro method of claim 11, wherein the recombinant protein is a hormone, a cytokine, a lymphokine, a fusion protein, an antibody, antigen-binding fragment thereof, or an antibody protein product.

13. The in vitro method of any one of the preceding claims, wherein the therapeutic protein is present in the mixture at a final concentration of about 10 μg/mL to about 300 μg/mL, optionally, greater than about 100 μg/mL or greater than about 200 μg/mL.

14. The in vitro method of any one of the preceding claims, wherein the mixture comprises greater than about 87% (v/v) serum or depleted serum at the beginning of step (a), optionally, greater than about 90% (v/v) serum or depleted serum, such as about 92% to about 98% (v) serum or depleted serum.

15. The in vitro method of any one of the preceding claims, wherein the depleted fraction of serum is an IgG-depleted serum fraction, optionally, obtained by removing IgG from serum by Protein A affinity chromatography.

16. The in vitro method of any one of the preceding claims, wherein the depleted fraction of serum is a fraction depleted of molecules having a pre-selected molecular weight range, optionally, wherein the pre-selected molecular weight range is about 30 kDa to about 300 kDa or higher, optionally, wherein the depleted fraction is obtained by through size-based filtration.

17. The in vitro method of any one of the preceding claims, wherein the depleted fraction is a twice-depleted fraction, optionally, a fraction twice-depleted of IgG or a fraction twice-depleted of molecules having a pre-selected molecular weight.

18. The in vitro method of any one of claims 1-14, wherein the mixture comprises whole serum.

19. The in vitro method of claim 18, wherein the whole serum is human serum, bovine serum, rabbit serum, mouse serum, rat serum, cyno serum, horse serum, or pig serum.

20. The in vitro method of claim 19, wherein the whole serum is human serum.

21. The in vitro method of any one of claims 18-20, wherein (i) the sample comprises therapeutic proteins comprising a fluorescent label or (ii) the method further comprises labeling the therapeutic proteins with a fluorescent label prior to step (a).

22. The in vitro method of claim 21, wherein the fluorescent label is selected from the group consisting of fluorescein, rhodamine, green fluorescent protein (and variants thereof), etc.

23. The in vitro method of any one of claims 20-22, further comprising a dilution step after step (a) and before step (b), optionally, wherein the mixture is diluted with water or buffer prior to step (b).

24. The in vitro method of any one of the preceding claims, wherein step (b) comprises assaying the level of HMW species in the mixture, which comprises serum or a depleted fraction thereof, by SEC.

25. The in vitro method of any one of the preceding claims, wherein the SEC is SEC-high performance liquid chromatography (SEC-HPLC) or SEC Fluorescence (SEC-Fluor) or SEC-UV.

26. The in vitro method of any one of the preceding claims, further comprising separating components of the mixture after step (a) and before step (b).

27. The in vitro method of claim 26, wherein the components are separated by chromatography, optionally, affinity chromatography.

28. The in vitro method of claim 27, wherein the affinity chromatography is affinity chromatography with Protein A, Protein L, or an antibody specific for the therapeutic protein.

29. The in vitro method of claim 28, wherein the affinity chromatography comprises an elution step comprising eluting with an acidic elution buffer.

30. The in vitro method of claim 29, wherein the acidic elution buffer comprises glycine or acetic acid or citrate.

31. The in vitro method of claim 31, wherein the acidic elution buffer has a pH of about 2.5 to about 4.5, optionally, about 2.75 to about 4.0.

32. The in vitro method of claim 31, wherein the pH is about 3.0 to about 4.0.

33. The in vitro method of any one of claims 29-32, wherein the elution step yields an eluate comprising the therapeutic protein and the method comprises assaying the level of HMW species of the therapeutic protein present in the eluate.

34. The in vitro method of any one of claims 28-33, wherein a resin linked to Protein A, Protein L, or an antibody specific for the therapeutic protein is incubated with the mixture for less than 1 hour.

35. The in vitro method of claim 34, wherein a resin linked to Protein A, Protein L, or an antibody specific for the therapeutic protein is incubated with the mixture for less than 30 minutes.

36. The in vitro method of claim 35, wherein a resin linked to Protein A, Protein L, or an antibody specific for the therapeutic protein is incubated with the mixture for less than 20 minutes.

37. The in vitro method of claim 36, wherein a resin linked to Protein A, Protein L, or an antibody specific for the therapeutic protein is incubated with the mixture for less than about 15 minutes, optionally for about 5 minutes to about 10 minutes.

38. A method of determining the in vivo reversibility of HMW species of a therapeutic protein, comprising (A) assaying the in vivo level of high molecular weight (HMW) species of a therapeutic protein according to the in vitro method of any one of the preceding claims, wherein (i) the method further comprises assaying the level of HMW species present in the sample prior to the incubating step or (ii) the level of HMW species present in the sample prior to the incubating step is known and (B) comparing the level(s) of HMW species present in the mixture to the level of HMW species present in the sample prior to the incubating step.

39. A method of determining the in vivo reversibility of HMW species of a therapeutic protein, comprising:
a. incubating a mixture comprising a sample comprising the therapeutic protein and a depleted serum, wherein the depleted serum is a fraction depleted of molecules having a pre-selected molecular weight range, optionally, wherein the pre-selected molecular weight range is about 30 kDa to about 300 kDa or higher, optionally, wherein the depleted fraction is obtained through size-based filtration;
b. assaying the level of HMW species of the therapeutic protein present in the mixture at one or more time points after step (a) by SEC;
c. comparing the level(s) of the HMW species present in the mixture as assayed in step (b) to the level of the HMW species present in the sample prior to step (a); and
d. calculating the percentage of in vivo reversibility of the HMW species of the therapeutic protein.

40. The method of claim 39, wherein the therapeutic protein has a molecular weight of about 15 kDa or higher.

41. A method of determining the in vivo reversibility of HMW species of a therapeutic protein, comprising:
a. incubating a mixture comprising a sample comprising the therapeutic protein and a depleted serum, wherein the depleted serum is an IgG-depleted serum fraction, optionally, obtained by removing IgG from serum by Protein L- or Protein A-affinity chromatography;
b. separating components of the mixture by affinity chromatography with a capture molecule to obtain a fraction comprising the therapeutic protein and HMW species thereof;
c. assaying the level of HMW species of the therapeutic protein present in the fraction by SEC,
d. comparing the level(s) of the HMW species present in the fraction as assayed in step (c) to the level of the HMW species present in the sample prior to step (a); and
e. calculating the percentage of in vivo reversibility of the HMW species of the therapeutic protein.

42. The method of claim 41, wherein the capture molecule is Protein A and the therapeutic protein binds to Protein A, optionally, wherein the therapeutic protein is an antibody, an Fc fusion protein, or an antibody protein product comprising a Protein A binding site.

43. The method of claim 41 or 42, wherein step (b) comprises (i) loading the mixture onto an affinity chromatography column to obtain a bound fraction comprising the therapeutic protein and (ii) eluting the bound fraction off the column.

44. A method of determining the in vivo reversibility of HMW species of a therapeutic protein, comprising:
a. incubating a mixture comprising a sample comprising the therapeutic protein with whole serum, wherein the therapeutic protein comprises a fluorescent label;
b. diluting the mixture;
c. assaying the level of HMW species of the therapeutic protein present in the mixture at one or more time points after step (a) by SEC,
d. comparing the level(s) of the HMW species present in the mixture as assayed in step (c) to the level of the HMW species present in the sample prior to step (a); and
e. calculating the percentage of in vivo reversibility of the HMW species of the therapeutic protein.

45. A method of determining the in vivo reversibility of HMW species of a therapeutic protein, comprising:
a. incubating a mixture comprising a sample comprising the therapeutic protein and whole serum;
b. separating components of the mixture by affinity chromatography with a capture molecule to obtain a fraction comprising the therapeutic protein and HMW species thereof;
c. assaying the level of HMW species of the therapeutic protein present in the fraction by SEC,
d. comparing the level(s) of the HMW species present in the fraction as assayed in step (c) to the level of the HMW species present in the sample prior to step (a); and
e. calculating the percentage of in vivo reversibility of the HMW species of the therapeutic protein.

46. The method of claim 45, wherein the capture molecule is an antibody or a molecule other than an antibody, which binds to the therapeutic protein.

47. The method of claim 45 or 46, wherein step (b) comprises (i) loading the mixture onto an affinity chromatography column to obtain a bound fraction comprising the therapeutic protein and (ii) eluting the bound fraction off the column.

48. The method of any one of claims 39-47, wherein the percentage of in vivo reversibility of the HMW species of the therapeutic protein is calculated according to Equation 1.
